(19) United States
(12) Patent Application Publication
(10) Pub. No.: US 2016/0361088 A1
(43) Pub. Date: Dec. 15, 2016

(54) CATHETER WITH PRE-FORMED GEOMETRY FOR BODY LUMEN ACCESS

(71) Applicant: COVIDIEN LP, MANSFIELD, MA (US)

(72) Inventors: MARK A. MAGUIRE, HILLSBOROUGH, CA (US); JOHN O. MCWEENEY, BRIGHTON, MA (US); ALEXANDER A. LUBINSKI, SAN FRANCISCO, CA (US); CHRISTOPHER L. BAGLEY, SANTA CLARA, CA (US); EDWARD CARCAMO, SUNNYVALE, CA (US)

(21) Appl. No.: 15/179,305

(22) Filed: Jun. 10, 2016

Related U.S. Application Data
(60) Provisional application No. 62/315,792, filed on Mar. 31, 2016.
(60) Provisional application No. 62/174,686, filed on Jun. 12, 2015.

(51) Int. Cl. A61B 17/34; A61F 2/962; A61M 25/09

(52) U.S. Cl. CPC A61B 17/3415; A61M 25/09041; A61F 2/962; A61B 2017/3456; A61B 17/3478; A61B 2017/3482; A61B 2017/347; A61B 2017/00818

(57) ABSTRACT

Methods, apparatuses, and systems are described for accessing a body lumen for subsequent treatment thereof. Systems include a cannula configured to penetrate a wall of a body lumen and to passively transition to a nonlinear shape within the body lumen as an elongate member is withdrawn from the distal section of the cannula. Other embodiments include a handle, a cannula removably disposed within a lumen of the handle, and a penetration member sized to advance through a lumen of the cannula, the cannula configured to passively transition to a nonlinear shape as the penetration member is withdrawn from a distal section of the cannula, and a cannula hub configured to selectively engage with a proximal end of the handle to selectively lock rotational movement of the cannula with respect to the handle.

### CROSS REFERENCES

The present application for patent claims priority to U.S. Provisional Application Ser. No. 62/315,792 by Maguire et al., entitled “CATHETER WITH PRE-FORMED GEOMETRY FOR BODY LUMEN ACCESS,” filed Mar. 31, 2016, and U.S. Provisional Application Ser. No. 62/174,686 by Maguire et al., entitled “CATHETER WITH PRE-FORMED GEOMETRY FOR BODY LUMEN ACCESS,” filed Jun. 12, 2015, each assigned to the assignee hereof.

### BACKGROUND

Diseases and disorders of the gallbladder, pancreas, and bile ducts (i.e., pancreaticobiliary system) are associated with significant morbidity, mortality, and impaired quality of life. Obstructions, tumors, injuries, leakages, inflammation, infection and lesions can occur in these structures, which can eventually lead to conditions such as biliary colic, cholecystitis, choledocholithiasis, cholelithiasis, pancreatitis, pancreatic duct stone formations, and chronic abdominal pain. Diseases of the pancreaticobiliary system may also be associated with nutritional disorders, such as malnutrition, obesity, and high cholesterol.

To treat a biliary obstruction, an Endoscopic Ultrasound Guided Biliary Drainage (EUS-BD) procedure may be performed. In such a procedure, a clinician may advance an EUS endoscope into a patient's duodenum and then advance a needle from the endoscope through the duodenal wall or gastric wall and through the wall of the a bile duct. Once the bile duct has been accessed, a guide wire may be advanced through a needle within the endoscope and into the bile duct with the goal of directing the guide wire across the obstruction. In some cases, the needle is advanced from the endoscope through the duodenal wall and through the wall of the common bile duct proximal to an obstruction near the papilla of the duct, through the obstruction and finally through the ampulla of Vater and back into the duodenum.

However, the EUS-BD approach is complex, inherently risky to the patient, and there is a lack of tools specifically designed for the procedure. For example, needles used to pierce the wall of a bile duct (e.g., a fine needle aspiration (FNA) needle) are typically straight and rigid. Accordingly, once the needle pierces the bile duct, the guide wire will naturally advance into the duct in whichever direction the needle is pointing, which may be in a non-preferred direction or directly into the duct wall, thereby increasing the duration of the procedure and trauma to the patient when trying to urge the guide wire in the desired direction. Also, because the needle is straight, it may easily slip back through the pierced access hole in the duct, thereby requiring the clinician to re-pierce the duct, which may lead to increased biliary leakage from the duct into the retroperitoneal space or pancreatitis. In some cases, the sharpened end of the needle may bind the guide wire and cause difficulty moving the guide wire and remove internal devices. can shear off a portion of the guide wire, within the duct, causing serious procedural complications and emergency surgery. Additional complications with present systems include preventing biliary leakage after penetrating the common bile duct, advancing the guide wire across a biliary obstruction, and navigating the guide wire through tight anatomical tortuosity.

### SUMMARY

The described features generally relate to methods, devices, and systems for accessing and navigating body lumens for subsequent treatment thereof. In accordance with various embodiments, a system for providing access to a body lumen is provided. The system includes a cannula having an elongate tubular body with a proximal section having a proximal end, a distal section having a distal end, and a cannula lumen extending from the proximal end to the distal end of the cannula. The distal section of the cannula may be configured to penetrate a wall of the body lumen and passively transition to a nonlinear shape within the body lumen as an elongate member is withdrawn from the distal section of the cannula lumen.

As described with reference to several embodiments, an orientation of the distal end of the cannula is adjustable within the body lumen. In certain aspects, the cannula is configured to transmit torque from the proximal end of the cannula to the distal end of the cannula to rotate the distal end of the cannula within the body lumen. In addition, an angle of sweep of the nonlinear shape may be adjustable within the body lumen. The nonlinear shape of the cannula may be configured to retain the distal section of the cannula within the body lumen in order to avoid accidental loss of cannulation of the lumen. In certain embodiments, the nonlinear shape is pre-defined by a heat treatment process.

In some embodiments, the elongate member comprises a sharpened distal end and is configured to protrude from the distal end of the cannula to penetrate the wall of the body lumen. In certain aspects, the distal end of the cannula may be blunt, beveled, or sharpened.

The described system may further include a guide wire sized to advance through the cannula lumen and having at least a first portion with a first stiffness and a second portion with a second stiffness, such that the second stiffness is greater than the first stiffness. In such embodiments, the first stiffness is less than a stiffness of the distal section of the cannula, and the second stiffness is greater than the stiffness of the distal section of the cannula such that as the second portion of the guide wire is advanced and withdrawn past the distal section of the cannula, an angle of sweep of the nonlinear shape is adjusted.

The described system may also include a guide wire sized to advance through the cannula lumen and having a portion configured to transition the distal section of the cannula from the curved shape into a substantially linear shape to facilitate advancement of the guide wire further into the body lumen and/or to facilitate removal of the distal section of the cannula from the body lumen over the guide wire.

According to various embodiments, the cannula includes a plurality of apertures disposed along a length of the distal section that allow the distal section to be relatively more flexible than the proximal section. In certain aspects, the plurality of apertures are limited to the length of distal section that is configured to transition into the nonlinear shape. In another embodiment, the plurality of apertures extend proximally beyond the distal section that is curved, allowing the relatively flexible section of the cannula to extend into some portion of the straight portion of the cannula.

The plurality of apertures may be arranged in a first row of apertures extending longitudinally along the length of the distal section and a second row of apertures extending longitudinally along the length of the distal section and disposed diametrically opposite the first row of apertures.

In some embodiments, the plurality of apertures are dogbone apertures having a central portion and an end portion on each end of the central portion, the central portion oriented substantially perpendicular to a longitudinal axis defined by the cannula lumen and each end portion oriented substantially perpendicular to the central portion. In other embodiments, the plurality of apertures are rectangular apertures oriented substantially perpendicular to a longitudinal axis defined by the cannula lumen. In yet other embodiments, the plurality of apertures are apertures having a plurality of curved features such as S-cut apertures.

As described with reference to several embodiments, an angle of sweep of the nonlinear shape is between 0 and 480 degrees. Additionally, the centerline radius of curvature of the nonlinear shape may range between 0.20 inches and 0.65 inches. In certain embodiments, an outer diameter of the cannula ranges between 0.02025 inches and 0.065 inches.

Another embodiment of a system for providing access to a body lumen is provided. The system includes a handle having a proximal end, a distal end, and a handle lumen extending from the proximal end to the distal end of the handle. The system may also include a cannula removably disposed within the handle lumen, the cannula having an elongate tubular body having a proximal section having a proximal end, a distal section having a distal end, and a cannula lumen extending from the proximal end to the distal end of the cannula. Certain embodiments include a penetration member sized to advance through the cannula lumen, such that the distal section of the cannula is configured to passively transition to a nonlinear shape within the body lumen as the penetration member is withdrawn from the distal section of the cannula lumen. The system may also include a cannula hub coupled with the proximal end of the cannula, the cannula hub configured to facilitate rotation of the cannula. In certain embodiments, the cannula hub is configured to selectively engage with the proximal end of the handle to selectively lock rotational movement of the cannula with respect to the handle.

The described system may also include a sheath coupled with the distal end of the handle and having a sheath lumen sized to slidably accept the cannula.

In accordance with various embodiments, a method for accessing a body lumen is provided. The method may include maneuvering a cannula in proximity to the body lumen, the cannula having an elongate tubular body, the elongate tubular body having a proximal section having a proximal end, a distal section having a distal end, and a cannula lumen extending from the proximal end to the distal end of the cannula. The method may further include advancing a penetration member distally until a distal end of the penetration member protrudes from the distal end of the cannula. Certain embodiments of the method include accessing the body lumen by simultaneously advancing the cannula and the penetration member through a wall of the body lumen and then withdrawing the penetration member proximally, such that the distal section of the cannula passively transitions into a nonlinear shape within the body lumen as the penetration member is withdrawn from the distal section of the cannula.

In certain embodiments, the method includes rotating the distal end of the cannula within the body lumen. Additionally or alternatively, the method includes advancing a guide wire through the cannula lumen and into the body lumen. In such embodiments, the method also includes adjusting an angle of sweep of the nonlinear shape by advancing a portion of the guide wire with a stiffness greater than a stiffness of the distal section of the cannula through the distal section of the cannula.

As described with reference to various embodiments, the nonlinear shape prevents the cannula from falling out of the body lumen. In some embodiments, the plurality of apertures extend long the straight portion of the cannula proximal to the pre-shaped distal section. Some embodiments further include aspirating fluid from the body lumen and injecting radio-opaque fluid through the cannula lumen and into the body lumen. In addition, some embodiments include the distal end of the sheath being configured to be advanced over the cannula into the body lumen to dilate the tract, for example. Moreover, the sheath may be a stent delivery catheter, whereby a biliary stent or similar device is delivered over the cannula and at least partially within the body lumen.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the embodiments may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a system for accessing a body lumen in accordance with aspects of the present disclosure;

FIG. 2A illustrates a system for accessing a body lumen with a distal section of a cannula in a generally linear configuration in accordance with aspects of the present disclosure;

FIG. 2B illustrates a system for accessing a body lumen with a distal section of a cannula in a generally nonlinear configuration in accordance with aspects of the present disclosure;

FIG. 2C illustrates the geometric parameters of a distal section of a cannula in a nonlinear configuration in accordance with aspects of the present disclosure;

FIG. 3 illustrates a system for accessing a body lumen piercing the wall of the body lumen in accordance with aspects of the present disclosure;

FIGS. 4A-4B illustrate a distal section of a cannula rotating within a body lumen in accordance with aspects of the present disclosure;

FIGS. 5A-5B illustrate a distal section of a cannula straightening out within a body lumen by advancement of a guide wire in accordance with aspects of the present disclosure;

FIGS. 6A-6B illustrate a cannula being advanced into a body lumen over a guide wire in accordance with aspects of the present disclosure;

FIGS. 7A-7D illustrate a sheath being advanced into a body lumen in accordance with aspects of the present disclosure;

FIGS. 8A-8G illustrate various aperture patterns disposed along a distal section of a cannula in accordance with aspects of the present disclosure;

FIGS. 9A-9C illustrate various angles of sweep of a distal section of a cannula in accordance with aspects of the present disclosure;

FIGS. 10A-10C illustrate various features of the distal end of a cannula in accordance with aspects of the present disclosure;

FIGS. 11A-1C illustrate various features of the distal end of a stylet in accordance with aspects of the present disclosure;

FIGS. 12A-12B illustrate a cannula being withdrawn back into an external sheath in accordance with aspects of the present disclosure;

FIG. 13 illustrates an exploded assembly view of a cannula hub and a handle member in accordance with aspects of the present disclosure;

FIG. 14 illustrates a system for accessing a body lumen of the pancreaticobiliary system in accordance with aspects of the present disclosure;

FIG. 15 illustrates a system for accessing a body lumen of the pancreaticobiliary system in accordance with aspects of the present disclosure;

FIG. 16 illustrates a flowchart of a method for accessing a body lumen in accordance with aspects of the present disclosure;

FIG. 17 illustrates a flowchart of a method for manipulating a cannula within a body lumen in accordance with aspects of the present disclosure;

FIG. 18 illustrates a flowchart of a method for accessing a body lumen in accordance with aspects of the present disclosure; and

FIG. 19 illustrates a flowchart of a method for accessing a body lumen in accordance with aspects of the present disclosure.

### DETAILED DESCRIPTION

The present disclosure is generally directed to apparatuses, systems, and methods for accessing a body lumen and navigating one or more treatment or diagnostic elements through the body lumen in a direction-controlled manner for subsequent treatment thereof. In accordance with various embodiments, a system for accessing a body lumen may include a cannula with a distal section configured to transition between a linear and nonlinear configuration. The system may also include a stylet and a guide wire, each configured to advance through a lumen of the cannula and to work in concert with the cannula to pierce the luminal wall and to manipulate the orientation of the distal end of the cannula within the lumen. In some embodiments, the system also includes a handle member with one or more features configured to manipulate the axial and rotational movement of the cannula, stylet, and guide wire with respect to each other and the accessed body lumen.

The cannula and stylet may be configured to function cooperatively to pierce the luminal wall for access into the body lumen. As described with reference to various embodiments, each of the cannula or stylet may include one or more features designed to reduce the piercing force required to pierce the luminal wall. For example, the distal ends of the cannula or stylet may be beveled, sharpened, or include energy-based cutting features. In addition, the cannula or stylet may include one or more features designed to coaxially align the stylet with the cannula as the two members pierce through tissue. In certain aspects, the cannula or stylet includes one or more features to gradually increase the diametrical profile between the stylet and cannula as the cannula follows behind the stylet to pierce the luminal wall.

Once the body lumen has been accessed, the distal section of the cannula may function to prevent the cannula from inadvertently slipping back through the pierced access hole in the lumen. As described with reference to various embodiments, the distal section of the cannula may transition into a nonlinear shape or configuration (e.g., a curved, arcuate, or looped shape), thereby anchoring the cannula within the lumen. In some examples, the distal section is pre-shaped through a memory shaping process such that the distal section of the cannula passively transitions into a nonlinear configuration in the absence of constraining forces (e.g., as a relatively more rigid internal stylet or external sheath is withdrawn from the distal section).

In various embodiments, the distal section includes a plurality of apertures to increase the flexibility of the distal section. As described with reference to various figures, the shape, size, and pattern of the apertures may be tailored to achiever certain cannula characteristics such as the shape of the distal section in the nonlinear configuration (e.g., angle of curvature and angle of sweep), a certain stiffness of the distal section (e.g., resistance to being pulled back through the pierced access hole), a particular flexibility profile (e.g., uniform or variable), or resistance to fracture (e.g., from local stress risers or fatigue loading). The apertures may be sized and located along the cannula to facilitate the advancement of the internal stylet or guide wire without catching on the apertures. In some embodiments, the same or different shaped apertures may be included on a section of the cannula proximal to the pre-curved distal section in order to provide flexibility along this section to facilitate advancement of the cannula deeper into the body lumen.

In addition to providing an anchoring force within the body lumen, the distal section of the cannula may be configured to change shape or orientation within the body lumen. For example, the cannula may be configured to transmit torque from the proximal end to the distal end, thereby allowing a clinician to rotate the distal section within the body lumen to change the orientation of the distal end with respect to the lumen (e.g., from retrograde to antegrade direction or vice versa). In addition, the angular orientation of the distal end of the cannula with respect to the lumen (i.e., the guidewire exit angle) may be changed within the body lumen by advancing a variable-stiffness guide wire through the cannula.

Embodiments of the present disclosure are now described in detail with reference to the drawings. As used herein, the term “clinician” refers to a doctor, surgeon, nurse, or any other care provider and may include support personnel. The term “proximal” will refer to the portion of the device or component thereof that is closer to the clinician and the term ‘distal” will refer to the portion of the device or component thereof that is farther from the clinician.

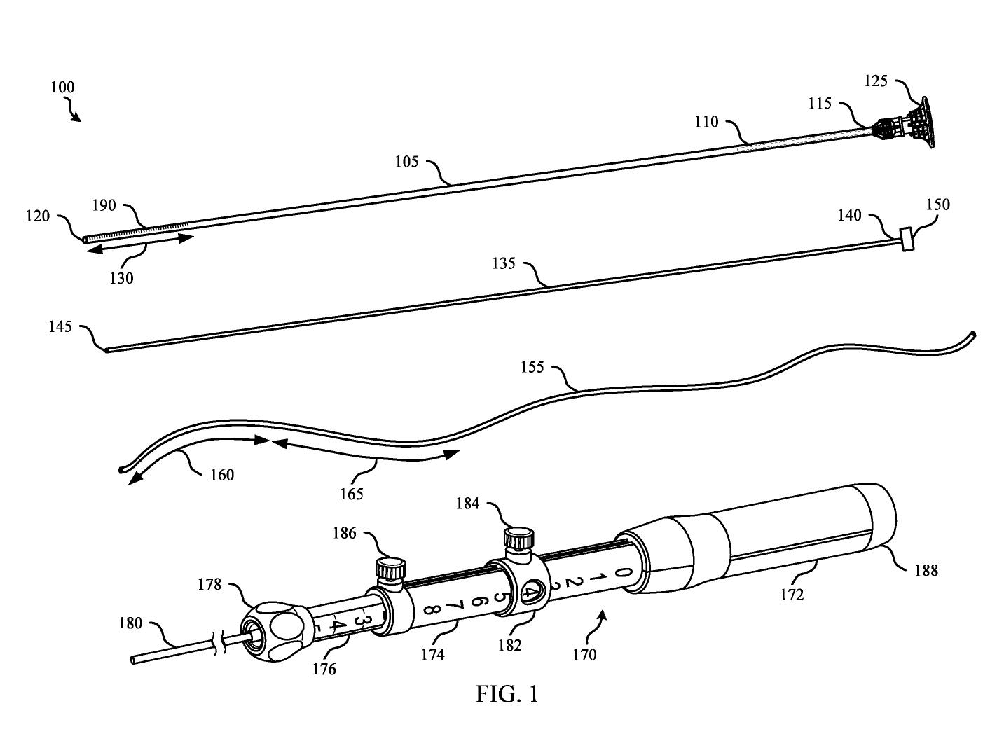

With reference to FIG. 1, an exploded view of a system 100 for providing access to a body lumen is illustrated in accordance with various embodiments. The system 100 generally includes a cannula 105, a stylet 135, a guide wire 155, and a handle assembly 170.

The system 100 can be provided as individual components, selectively combined components, or all together as a kit of components. The cannula 105 may be inserted into the handle assembly 170 (through the proximal end 188) until the cannula hub 125 abuts against the proximal end 188. Once assembled, the cannula 105 extends through the handle assembly 170 and through the sheath 180 to the target body lumen. During a luminal access procedure, the stylet 135 and guide wire 155 may be inserted into the cannula through the hub 125 (at different times) and advanced through the lumen 110 of the cannula 105. The system 100 may be used to access and provide treatment to one or more body lumens within the gastrointestinal system or pancreaticobiliary system, for example. It may be appreciated that the system 100 may also be used to provide access or treatment to other organs or luminal systems within the body such as the arterial system, the bronchial system, the urinary system, or any other luminal system were maneuverability and accuracy is desirable.

In some embodiments described herein, the handle 170 is coupled with an endoscope and the cannula 105 is guided via endoscopic ultrasound (EUS) to provide access to one or more body lumens or organs associated with the pancreaticobiliary system for the purpose of providing treatment. For example, the system 100 may be configured to provide access to at least the common biliary duct to facilitate subsequent procedures to treat narrowed areas or blockages within the bile duct, including palliative drainage procedures. In accordance with various embodiments, the system 100 may be used to perform an Endoscopic Ultrasound Guided Biliary Drainage (EUS-BD) procedure. In a particular embodiment, a palliative drainage procedure may be performed in antegrade fashion in conjunction with the access system 100. In another embodiment, the palliative drainage procedure may be performed in retrograde fashion, referred to as an Endoscopic Retrograde Cholangiopancreatography (ERCP) “Rendezvous” procedure.

The cannula 105 of the system 100 has an elongate tubular body and an internal lumen 110 extending from its proximal end 115 to the distal end 120. In general, the cannula 105 is configured to access a body lumen (e.g., by piercing a luminal wall) and to provide a conduit through which one or more devices (e.g., a guide wire 155) may pass to facilitate subsequent treatment of the body lumen or associate organs. As described with reference to several embodiments, the cannula 105 may include features that facilitate the direction-controlled delivery of a guide wire 155 within the body lumen for subsequent delivery of a stent, a biopsy device, a medicinal delivery element, or any number of other treatment or diagnostic devices.

The cannula 105 may be dimensioned to advance through the working channel of an endoscope (e.g., an EUS endoscope). To access an internal body lumen, a clinician may insert the cannula 105 into the working channel of an endoscope and advance the cannula 105 distally by pushing it from the proximal end 115 or cannula hub 125 of the cannula 105. Accordingly, the cannula 105 as described herein is configured to exhibit sufficient pushability (i.e., columnar strength) to be advanced through an endoscope and into a target location within the body. It may be appreciated that the pushability of the cannula 105 depends on the material stiffness and one or more dimensions (e.g., wall thickness, total length) of the cannula 105. In a particular embodiment, the cannula 105 has an outer diameter (OD) of approximately 0.0465 inches, an inner diameter (ID) (i.e., diameter of lumen 110) of approximately 0.0365 inches, and a length of approximately 70 inches. However, it should be appreciated that larger or smaller diameters or lengths may be used in accordance with various embodiments described herein to access body lumens of various sizes. For example, the outer diameter of the cannula 105 may be as small as 0.0131 inches (1 F) or as large as 0.0656 inches (5 F), and the ID, length, and material properties of the cannula 105 may be modified accordingly to maintain the pushability of the cannula 105.

The cannula 105 may be manufactured from a variety of materials such as a nickel-titanium alloy (i.e., nitinol) or a number of other metallic-based or polymeric-based materials. Exemplary metallic materials include, but are not limited to, stainless steel, such as 304V, 304L, and 316L stainless steel; linear-elastic or super-elastic nitinol or other nickel-titanium alloys, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; and cobalt chromium alloys. Exemplary polymeric-based materials include, but are not limited to, poly-ether-ether ketone, polyamide, poyethersulfone, polyurethane, ether block amide copolymers, polyacetal, polytetrafluoroethylene or derivatives thereof.

In certain embodiments, the distal end 120 of the cannula 105 is blunt (i.e., free from beveled or sharpened features) so as to prevent catching, gouging, or piercing as the cannula 105 is advanced through an outer sheath (e.g., the working channel of an endoscope or an outer sheath 180). The blunt distal end 120 may also prevent the guide wire 155 or any other element that is advanced from the distal end 120 from being pinched from free movement within the cannula 105, or portions sheared off inside the target body lumen, thereby improving the safety of the system 100. Alternatively, the distal end 120 of the cannula 105 may be beveled without having sharp cutting edges, or can instead be sharpened for piercing tissue such as the wall of a body lumen.

As described with reference to various embodiments herein, a distal section 130 of the cannula 105 may be configured to passively transition (i.e., naturally move in the absence of constraining forces) from a linear shape into a nonlinear shape within a body lumen, and vice versa. For example, in accordance with various embodiments, the distal section 130 may be constrained in a linear configuration by an internal member (e.g., a stylet 135) or an external member (e.g., a sheath 180), and as the member is withdrawn (e.g., translated axially with respect to the cannula 105 in the proximal direction), the distal section 130 may then passively transition into a nonlinear configuration. In contrast, manipulating the distal section 130 manually into a nonlinear configuration with a pull wire or other force-transmitting component would be an example of a non-passive (i.e., active) transition.

In accordance with various embodiments, one or more apertures 190 are disposed along the distal section 130 and are sized, arranged, or otherwise configured to facilitate flexing or bending of the distal section 130 to the nonlinear shape. As described herein, a variety of shapes and patterns of the apertures 190 may be used to impart certain flexibility characteristics to the cannula 105. In some examples, the apertures 190 may extend proximal to distal section 130, thereby providing flexibility to portions of the cannula 105 that are not configured to passively transition into a nonlinear shape. The nonlinear shape of the distal section 130 may act as an anchor within the body lumen, thereby preventing the cannula 105 from inadvertently falling back out of the body lumen. This anchoring feature may be advantageous during exchange maneuvers, such as when a stylet 135 is withdrawn from the cannula and replaced with a guide wire 155. In addition to making the procedure easier for a clinician, preventing cannula fallout may reduce the need to re-pierce the body lumen, thereby reducing trauma and risk to the patient.

Additionally, the orientation, position, size, or geometry of the distal section 130 may be adjusted within the body lumen to provide directional control over the placement and advancement of the guide wire 155 (or any other treatment or diagnostic device) within the body lumen. Such directional control may allow a clinician to advance the guide wire 155 in a preferred direction within the body lumen regardless of the orientation of the distal end 120 of the cannula 105 when it is initially advanced into the body lumen. As described with reference to various embodiments, a clinician may use any combination of rotation, straightening, bending, or longitudinal (i.e., axial) movement to adjust the orientation of the distal end 120 so that the guide wire 155 exits the cannula 105 in a preferred direction within the body lumen, as required by the particular medical procedure.

In accordance with various embodiments, the distal section 130 may be pre-shaped into a particular nonlinear shape such that the distal section 130 can be straightened into a linear configuration, but will passively return to the pre-shaped nonlinear configuration when unconstrained. For example, utilizing the heat-memory properties of nickel titanium alloy (i.e., nitinol), the distal section 130 can be heat set into the desired curved shape by holding the cannula 105 in the desired shape and heating to an appropriate temperature, then cooling again, thereby imparting a heat set memory to the material. The heating method can be an air or vacuum furnace, salt bath, sand bath, heated die, or other heating method.

An exemplary heat setting process may include heating the cannula 105 in the range of 500°-550° C., with higher temperatures resulting in lower tensile strengths. The cooling process should generally occur rapidly to avoid aging effects and may be performed with a water quench. The heat treatment time should be such that the material reaches the desired temperature throughout its cross-section. As may be appreciated, the time will depend on the mass of the fixture, the material of the cannula 105, and the heating method. Times may be less than a minute for heating small parts in a salt bath or heated die. Times may be much longer (e.g., 10-20 minutes) for heating massive fixtures in a furnace with an air or argon atmosphere. In these cases a thermocouple in contact with the material or fixture is recommended. In all cases, experimentation for the proper time and temperature will be required to determine the combination that gives the desired results.

Referring still to FIG. 1, the cannula 105 may include a cannula hub 125 coupled with the proximal end 115 of the cannula 105, which includes an internal lumen in fluid communication with the internal lumen 110 of the cannula 105, thereby allowing passage of devices (e.g., a stylet 135 or guide wire 155) or fluid through the hub 125 into the internal lumen 110. The cannula hub 125 (which is located outside of the body during a procedure) may be rotated about a longitudinal axis of the cannula 105 by a clinician to cause rotation of the distal end 120. As described with reference to various embodiments below, the cannula hub 125 may interface with a portion of the handle assembly 170 (e.g., proximal portion 188) to provide controlled rotation of the cannula hub 125 with respect to the handle assembly 170. This may include providing frictional resistance to rotation (e.g., with an O-ring interface) or by providing a selective locking and unlocking feature between the cannula hub 125 and the handle assembly 170. Such controlled rotation may advantageously allow a clinician to rotate the cannula 105 a desired amount and then hold the cannula hub 125 in place relative to the handle assembly 170, thereby preventing the cannula 105 from rapidly unwinding (i.e., whipping) or to otherwise prevent inadvertent movement of the distal section 130 within the body lumen.

The stylet 135 is generally an elongate, cylindrical member with proximal end 140 and distal end 145, and is dimensioned to slidably advance through the lumen 110 of the cannula 105. The stylet 135 is generally solid (i.e., no internal lumen), but may include an internal lumen in some embodiments (e.g., FNA needle). The stylet 135 may also have a non-circular cross section (e.g., triangle, square), thereby allowing more space for fluid to be injected or aspirated through the cannula lumen 110 surrounding the stylet 135. The stylet 135 may also include a hub 150 coupled with the proximal end 140 of the stylet 135 to facilitate longitudinal or rotational manipulation of the stylet 135 with respect to the cannula 105. In certain embodiments, the distal end 145 of the stylet 135 is sharpened or otherwise configured to pierce bodily tissue such as the duodenal wall and the wall of a target organ or vessel, such as the common bile duct. To pierce tissue (e.g., a luminal wall), the distal end 145 of the stylet 135 may be advanced from the distal end 120 of the cannula 105, thereby exposing the sharpened (or energizable) distal end 145 of the stylet 135. Once the distal end 145 of the stylet 135 is exposed, the stylet 135 and cannula 105 may be advanced simultaneously to pierce through a luminal wall or other target tissue. As described with reference to various embodiments, the distal end 145 of the stylet 135 may be sharpened (e.g., by grinding) into a variety of configurations such as a hypodermic grind, a trochar grind, or a four-plane grind, for example.

For a variety of reasons, it may be desirable to minimize the diametrical transition from the outer diameter of the stylet 135 to the outer diameter of the cannula 105. For example, the cannula 105 or the stylet 135 may include one or more tapered features to smooth the transition from the outer diameter of the stylet 135 to the outer diameter of the cannula 105, thereby reducing the force required to pierce through tissue.

The guide wire 155 is generally a flexible elongate member configured to slidably advance through the lumen 110 of the cannula 105. The guide wire 155 may be uniform in size and stiffness along its entire length, or alternatively, may include sections of differing stiffness. For example, a distal section 160 of guide wire 155 may be less stiff (i.e., more floppy) than a more proximal section 165. Although two sections 160, 165 are shown, it may be appreciated that a guide wire 155 with more sections may be used. As described in more detail below, a clinician may exploit the variable stiffness between sections 160, 165 to adjust a geometry of the distal section 130 of the cannula 105 within the body lumen (e.g., by straightening out the nonlinear shape). As such, the number, length, and relative stiffness of sections 160, 165 (or additional sections) may be selected to provide a particular amount of straightening (of distal section 130) for a particular total length of guide wire 155 within the body lumen. The guide wire 155 may be made from a variety of flexible materials, including but not limited to nitinol, stainless steel, platinum, gold or other suitable metals. In addition, the guide wire 155 may be comprised of a metallic core surrounded by a polymeric outer jacket. In a particular embodiment, the outer diameter of the guide wire 155 is approximately 0.035 inches, but other diameters may be used depending on the size of the cannula 105 being used or the size of the body lumen being accessed.

The handle assembly 170 is generally configured to facilitate manipulation of the cannula 105, the stylet 135, and the guide wire 155 with respect to each other, the accessed body lumen, or an attached endoscope. The handle assembly 170 may include a proximal handle member 172 with a proximal portion 188, a middle handle member 174, and a distal handle member 176. The proximal, middle, and distal handle members 172, 174, 176 each include an inner lumen and are coupled together to form a continuous lumen extending throughout the length of the handle assembly 170. The proximal handle member 172 is slidably disposed over at least a portion of the middle handle member 174, and, similarly, the middle handle member 174 is slidably disposed over at least a portion of distal handle member 176. The distal handle member 176 may also include a threaded connector element 178 configured to securely attach to a working channel of an endoscope (not shown).

The handle assembly 170 may also include a sheath 180 extending from the distal end of the distal handle member 176. The sheath 180 is generally made from a flexible polymeric material and provides a continuous conduit through which the cannula 105 or other elements may travel between the handle assembly 170 and the target tissue within the body (e.g., the bile duct). Accordingly, the length and diameter of the sheath 180 depend upon the particular application. In some embodiments, the sheath 180 is braided and may include one or more features at the distal end that may be used by a clinician to straighten out the distal section 130 of the cannula 105. Also, in certain examples, the distal end of the sheath 180 may be tapered or include energy-based cutting features to facilitate the advancement of the sheath 180 over the cannula 105 into the body lumen.

The handle assembly 170 may also include one or more adjustment features that limit the sliding movement of the handle members 172, 174, 176 relative to each other. For instance, the handle assembly 170 may include a locking ring 182 with a threaded thumbscrew 184 disposed around the middle handle member 174. The locking ring 182 may be slid along the middle handle member 174 and tightened in a desired position with the thumbscrew 184. When tightened, the locking ring 182 limits the movement of the proximal handle member 172 in the distal direction relative to the middle handle member 174, thereby allowing the clinician to establish a set penetration depth of the cannula 105 or stylet 135 beyond the distal end of the sheath 180. Similarly, a thumbscrew 186 is configured to lock the position of the distal handle member 176 with respect to the middle handle member 174, thereby allowing the clinician to set an extension depth of the sheath 180 beyond the distal end of an attached endoscope.

The access assembly 100 may be compatible for use with exemplary endoscopic delivery systems and methods discussed in co-owned applications titled Needle Biopsy Device with Exchangeable Needle and Integrated Needle Protection (U.S. Pub. 2012/0116248), Rapid Exchange FNA Biopsy Device with Diagnostic and Therapeutic Capabilities (U.S. Pub. 2011/0190662), Device for Needle Biopsy with Integrated Needle Protection (U.S. Pub. 2010/0121218), or Needle Biopsy Device (U.S. Pub. 2010/0081965), the contents of which are hereby incorporated by reference in their entirety.

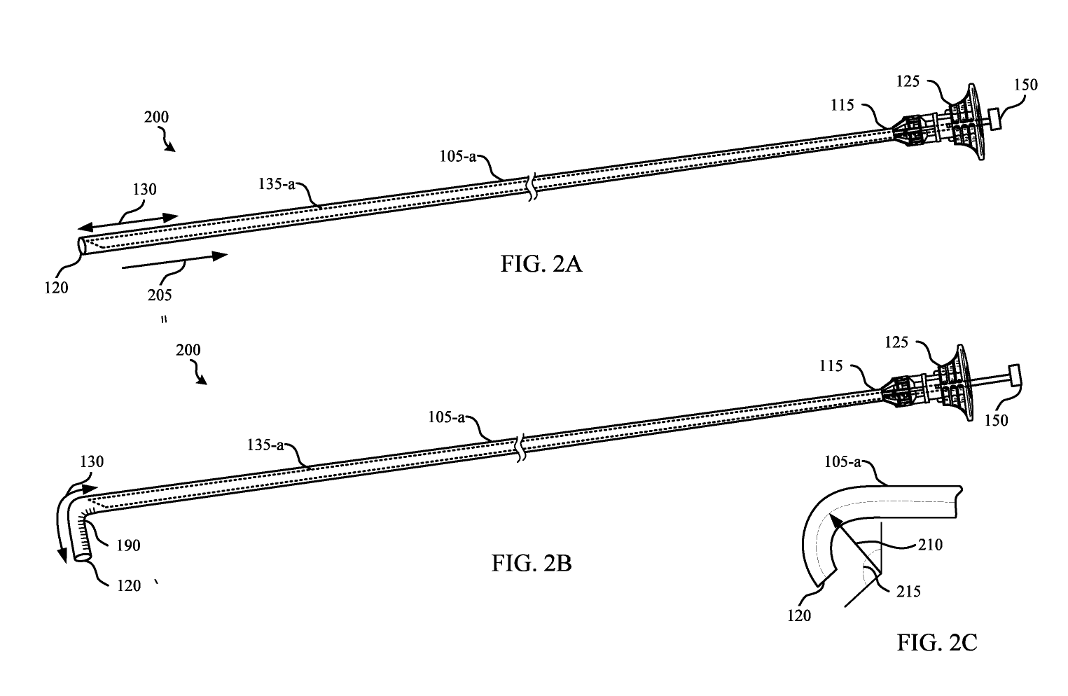

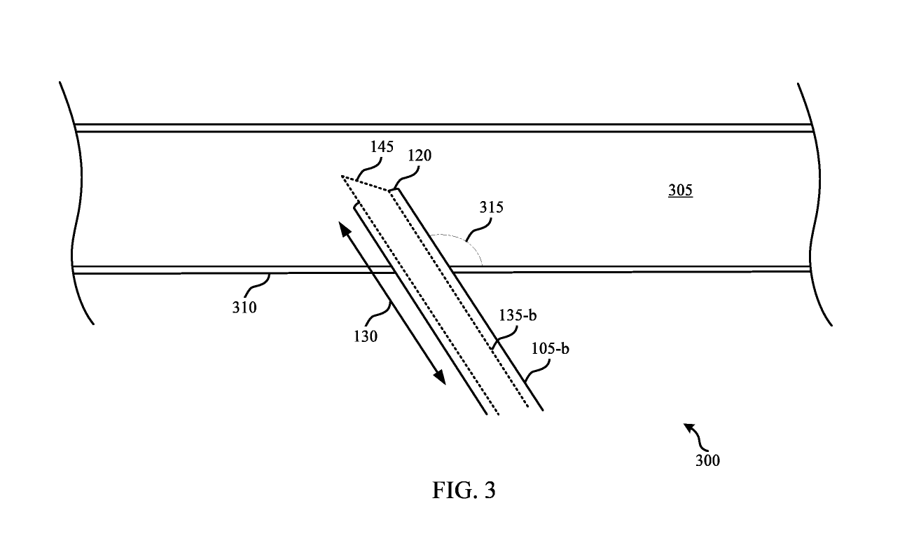

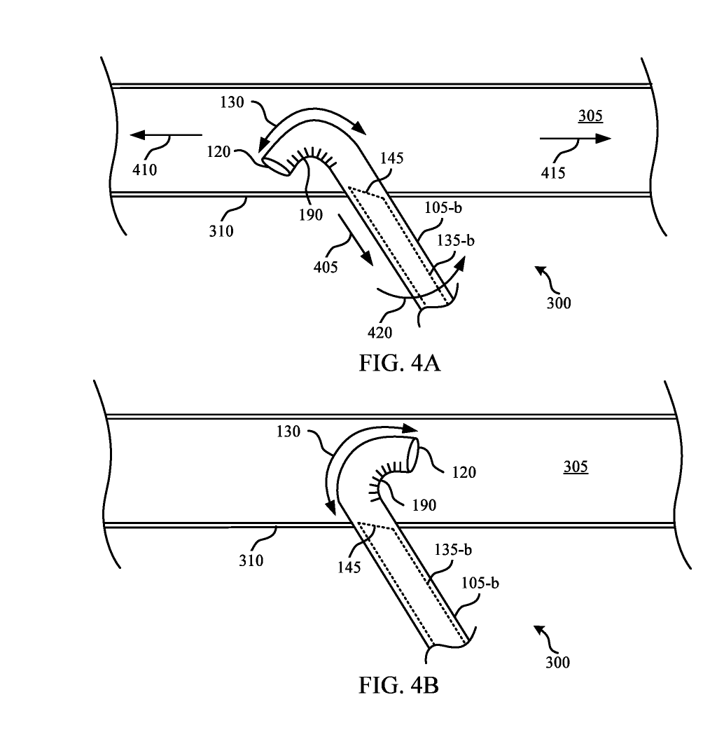

With reference to FIGS. 2A-2B, a schematic view of a system 200 for providing access to a body lumen is illustrated in accordance with various embodiments. The system 200 includes a stylet 135-a (shown in phantom lines) slidably disposed within the lumen 110 (not shown for clarity) of a cannula 105-a. The stylet 135-a and the cannula 105-a may be examples of the stylet 135 and the cannula 105 described with reference to FIG. 1. As illustrated in FIGS. 2A and 2B, the distal section 130 of the cannula 105-a may be configured to passively transition from a linear configuration (FIG. 2A) to a nonlinear configuration (FIG. 2B) as the stylet 135-a is withdrawn in a proximal direction 205 from the distal section 130 of the cannula lumen 110. As described with reference to FIG. 1, this passive transition may be achieved by shape setting the distal section 130 of the cannula 105-a into a pre-defined nonlinear shape such that in the absence of external constraining forces (i.e., upon removal of the relatively stiffer stylet 135-a), the distal section 130 will transition into the pre-defined nonlinear shape. It may be appreciated that the stiffness of the distal section 130 relative to the stiffness of the stylet 135-a may be adjusted such that the distal section 130 conforms to the shape of the stylet 135-a (e.g., linear) while the stylet 135-a is within the distal section 130. As shown, the distal section 130 may include a plurality of apertures 190 (shown only in FIG. 2B for clarity) that impart additional flexibility to the distal section 130.

Additionally or alternatively, the distal section 130 may be passively transitioned into a nonlinear configuration by advancing and retracting the distal section 130 in and out of an external sheath (e.g., sheath 180) even when the stylet 135-a is not within the distal section 130. In such examples, the external sheath provides the constraining forces that keep the distal section 130 from passively transitioning into a nonlinear shape.

With reference to FIG. 2C, in some embodiments, the nonlinear shape is a circular arc and may be defined by a centerline radius of curvature 210 and an angle of sweep 215 (i.e., the angular displacement of the distal end 120 from a linear configuration). Alternatively, the nonlinear shape may be a non-uniform arc (e.g., an elbow or non-circular arc) or may include a combination of arcs or other various nonlinear features with varying shape and sizes. In the embodiment illustrated in FIG. 2C, the angle of sweep 215 of the nonlinear shape is approximately 135°. In accordance with other embodiments described herein, the angle of sweep 215 may range anywhere from 0° (i.e., linear) to 480°. Exemplary embodiments described include an angle of sweep 215 of 45°, 90°, 135°, 180°, and 270°.

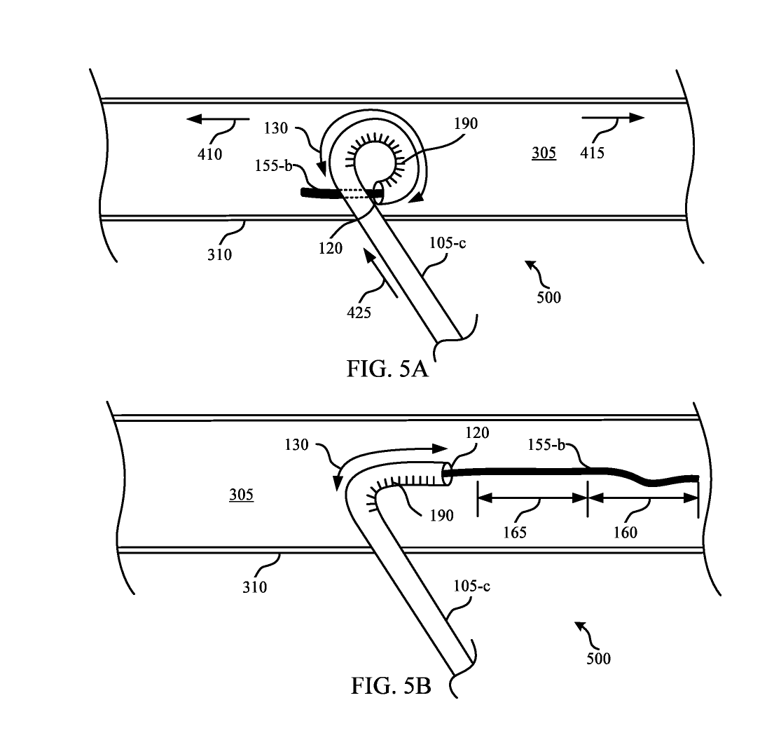

With reference to FIG. 3, a schematic view of a system 300 for providing access to a body lumen 305 is illustrated in accordance with various embodiments. The body lumen 305 may represent any lumen within the body such as those within the biliary system, the arterial system, the bronchial system, or the urinary system. The system 300 includes a stylet 135-b (shown in phantom lines) slidably disposed within a cannula 105-b, which may be examples of the cannula 105 and the stylet 135 described with reference to FIGS. 1-2. In the illustrated embodiment, the distal end 120 of the cannula 105-b is blunt whereas the distal end 145 of stylet 135-b is sharpened and configured to pierce the wall 310 of the body lumen 305. In an alternative embodiment, the distal end 120 of the cannula 105-b may be beveled but without sharp edges. In yet another embodiment, the distal end 120 of the cannula 105-b may be sharpened in order to facilitate piercing the lumen wall 310 with an internal stylet 135-b with a distal end 145 that is blunt and therefore not adapted to pierce tissue. In such examples, the stylet 135-b may provide columnar support to the cannula 105-b during piercing.

As the system 300 is being maneuvered through the body to the body lumen 305 (e.g., through the working channel of an endoscope or through an external sheath 180), the sharpened distal end 145 of the stylet 135-b may be retracted within the cannula 105-b so as to not protrude from the distal end 120. In this way, the distal end 145 of the stylet 135-b will be prevented from catching (e.g., gouging or scraping) on the internal surface of the working channel of the endoscope or the sheath 180. However, the stylet 135-b may be positioned within the distal section 130 of the cannula 105-b while the cannula 105-b is being maneuvered through the endoscope so as to prevent the distal section 130 from transitioning into a nonlinear configuration prematurely (i.e., before accessing the body lumen 305).

To access the body lumen 305, the distal end 145 of the stylet 135-b may be advanced distally so as to protrude from the distal end 120 of the cannula 105-b thereby exposing the sharpened distal end 145. The stylet 135-b can be axially fixed with respect to the cannula 105-b by releasably attaching the stylet 135-b to the proximal end 188 of the handle assembly 170, for example by engaging a luer feature on the stylet hub 150 with a complementary luer fitting on the proximal end 188 of the handle assembly 170. The cannula 105-b and stylet 135-b may then be advanced simultaneously to pierce the wall 310 of the body lumen 305. As described in further detail below, the shape of the sharpened distal end 145 (e.g., angle and number of beveled cuts) may be optimized to reduce the piercing force required to pierce through the luminal wall 310. Moreover, the gap (e.g., the difference between the outer diameter of the stylet 135-b and the outer diameter of the cannula 105-b) and coaxial alignment between the stylet 135-b and the cannula 105-b may be adjusted to further reduce the required piercing force.

In an alternative embodiment, the distal end 145 of the stylet 135-b includes an energizable (e.g., radiofrequency energy) element configured to cut, ablate, or otherwise penetrate through the wall 310 of the body lumen 305. For example, the distal end 145 may include a diathermic or dielectric cutting element including but not limited to a dielectric cautery ring, a cutting knife, a cutting wire, pinching cutters, or the like configured to allow the clinician to ablate or otherwise cut through tissue so as to widen an obstructed pathway or completely remove a tumor or other obstruction (e.g., gallstone). An energizable element may penetrate tissue less traumatically than a sharpened tip 145 and the cutting ability of the tip would stop when the energy is discontinued, thereby reducing the risk of inadvertent pierce (e.g., re-piercing the luminal wall 310 once inside the lumen 305). Alternatively, the energizable element could be integrated into the distal tip of the guide wire 155, thereby eliminating the need to exchange the stylet 135-b for the guide wire 155 because the guide wire 155 would provide both the piercing function and the support function for steps after gaining luminal access such as therapy device delivery.

As illustrated, the cannula 105-b may pierce the lumen 305 at a particular angle 315. For a variety of reasons (e.g., anatomical location of the body lumen, limits on maneuverability of endoscopic systems), it is difficult for a clinician to control or predict the angle 315 at which the cannula 105-b will pierce the body lumen 305. Thus, without the ability to reorient the direction of the distal end 120, the clinician is generally forced to deploy a guide wire 155 (or other internal element) from the cannula 105-b in the direction dictated by the pierce angle 315, which may be undesirable. For example, if the pierce angle 315 were approximately 90°, then the guide wire 155 would be deployed directly into the wall of the lumen 310 opposite the access hole, which may damage the lumen 305. In another example, the distal end 120 may initially point in the retrograde direction whereas the preferred direction may be antegrade. Conversely, in certain instances, the distal end 120 may initially point in the antegrade direction, but the retrograde direction may have been preferred to treat obstructions in the extra-hepatic or intra-hepatic biliary ducts. In accordance with various embodiments described herein, the cannula 105-b may include one or more features that facilitate a clinician to maneuver the distal end 120 within the body lumen 305 and to reorient the distal end 120 along a desired direction within the body lumen 305 regardless of the initial pierce angle 315.

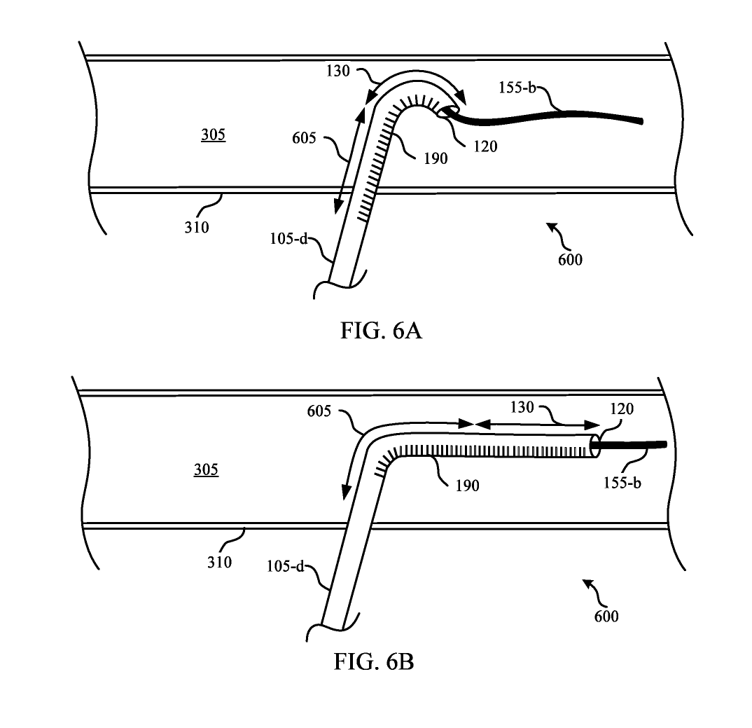

With reference to FIGS. 4A-4B, a schematic view of the system 300 from FIG. 3 is illustrated with the distal section 130 of the cannula 105-b transitioned into a nonlinear shape. As described with reference to FIG. 2A-2B, the distal section 130 of the cannula 105-b may be configured to passively transition from the linear shape (FIG. 3) to a nonlinear shape (FIG. 4A-4B) as the stylet 135-b is withdrawn in a proximal direction 405 from the distal section 130. It may be appreciated, however, that once the distal section 130 has transitioned into a nonlinear shape, the distal end 120 of the cannula 135-b may be pointing in a direction or otherwise orientated at an angle with respect to the lumen other than that desired for treatment. For example, the distal end 120 may be generally pointing in a direction 410, which may correspond with retrograde flow within the body lumen 305. Accordingly, if a guide wire 155 were advanced from the distal end 120, the guide wire 155 would naturally advance through the lumen 305 in the retrograde direction 410. For various reasons, it may be desirable instead to advance the guide wire 155 in the antegrade direction 415. Alternatively, the distal end 120 may be initially pointing in the antegrade direction 415, whereas the preferred direction for a particular procedure would have been in the retrograde direction 410.

In accordance with various embodiments, the orientation of the distal end 120 of the cannula 105-b is adjustable within the body lumen 305. For example, the cannula 105-b may be configured to transmit torque from the proximal end 115 to the distal end 120 so that the distal end 120 may be rotated within the body lumen 305 by a clinician. In the illustrated embodiment, the distal end 120 may be rotated (as indicated by arrow 420) about a longitudinal axis of the cannula 105-b until the distal end 120 points generally in the antegrade direction 415. It may be appreciated that the dimensions of the cannula 105-b (e.g., outer diameter, wall thickness, and length) as well as the material characteristics (e.g., stiffness) of the cannula 105-b are chosen to facilitate the transmission of torque from the proximal end 115 to the distal end 120. In a particular embodiment, the cannula 105-b is formed from a metal (e.g., nitinol) with appropriate material stiffness and wall thickness to provide torque and columnar strength that is optimized for the application in a relatively low profile (i.e., small outer diameter of cannula 105-b). Alternatively, the cannula 105-b may be made from a polymeric-based braid or coil-reinforced composite, although such a configuration may require a thicker cannula wall and therefore a larger outer diameter.

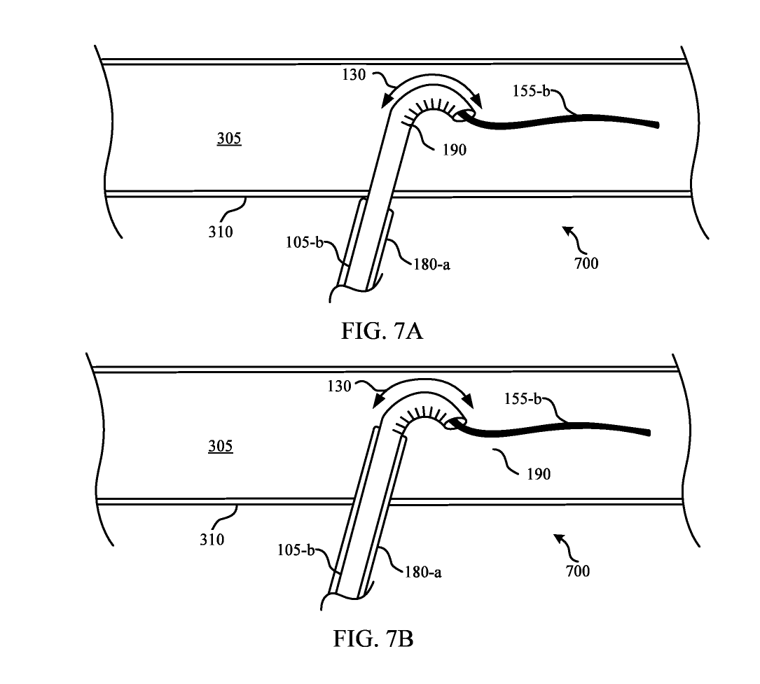

With reference to FIGS. 5A-5B, a schematic view of a system 500 for providing access to a body lumen 305 is illustrated in accordance with various embodiments. The system 500 includes a guide wire 155-b slidably disposed within a cannula 105-c, which may be examples of the cannula 105 and the guide wire 155 described with reference to any of the FIGS. 1-4. The cannula 105-c illustrated in FIG. 5A includes a distal section 130 that is already transitioned into a nonlinear shape. As shown, the angle of sweep 215 of the nonlinear shape exceeds 90°, and is approximately 270°. This configuration may be referred to as a cloverleaf, looped, or pigtail configuration. Such an angle of sweep 215 may advantageously retain the cannula 105-c within the body lumen 305 thereby preventing the distal section 130 from inadvertently falling out of the lumen 305. In addition, the illustrated nonlinear shape is relatively atraumatic because the side of the cannula 105-c is resting against the luminal wall 310 as opposed to the distal end 120.

As described with reference to FIGS. 4A-4B, a clinician may desire to change the orientation of the distal end 120 of the cannula 105-c within the body lumen 305, and may do so by rotating the cannula 105-c about its longitudinal axis. Additionally or alternatively, orientation of the distal end 120 may be manipulated by straightening out (or subsequently curling up) the distal section 130 of the cannula 105-c within the body lumen 305. For example, as illustrated in FIG. 5A, once the distal section 130 of the cannula 105-c has transitioned into a nonlinear shape, the distal end 120 may face generally along direction 410 (which may correspond to the retrograde flow within the body lumen 305). To reorient the distal end 120 so that it generally faces the opposite direction 415, the distal section 130 may be straightened out (i.e., reducing an angle of sweep 215) by manipulating the internal guide wire 155-b with respect to the cannula 105-c. It may be appreciated that the angle of sweep adjustment needed to align the distal end 120 in a preferred direction will depend on the angle of sweep 215 of the distal section 130 (e.g., 135° or 270°) as well as the angle at which the distal section 130 pierced the luminal wall 310. Thus, the ability to adjust the angle of sweep 215 within the body lumen 305 may be advantageous because it is typically difficult to predict or control the angle at which the cannula 105 will pierce the luminal wall 310 (as described with reference to FIG. 3).

In accordance with various embodiments, the distal section 130 is straightened out by advancing a portion of the guide wire 155-b that is stiffer than the distal section 130. For example, referring to FIG. 5B, distal section 160 of the guide wire 155-b may be less stiff than the distal section 130 (thereby having little to no effect on the distal section 130), but section 165 of the guide wire 155-b may be stiffer than distal section 130, such that as section 165 is advanced from the distal end 120, an angle of sweep 215 of the nonlinear shape is reduced (i.e., the distal section 130 begins to straighten out). In this way, the various stiffness transitions of the guide wire 155-b may be used to relax or tighten the angle of sweep 215 of the distal section 130. It may be appreciated that the lengths of sections 160, 165 may be adjusted to provide the desired amount of straightening in conjunction with the desired amount of guide wire 155-b dispensed within the body lumen 305. It may also be appreciated that a clinician may use a combination of rotation and straightening to control the orientation of the distal end 120 with respect to the body lumen thereby controllably directing the guide wire 155-b as it is being advanced into the body lumen 305.

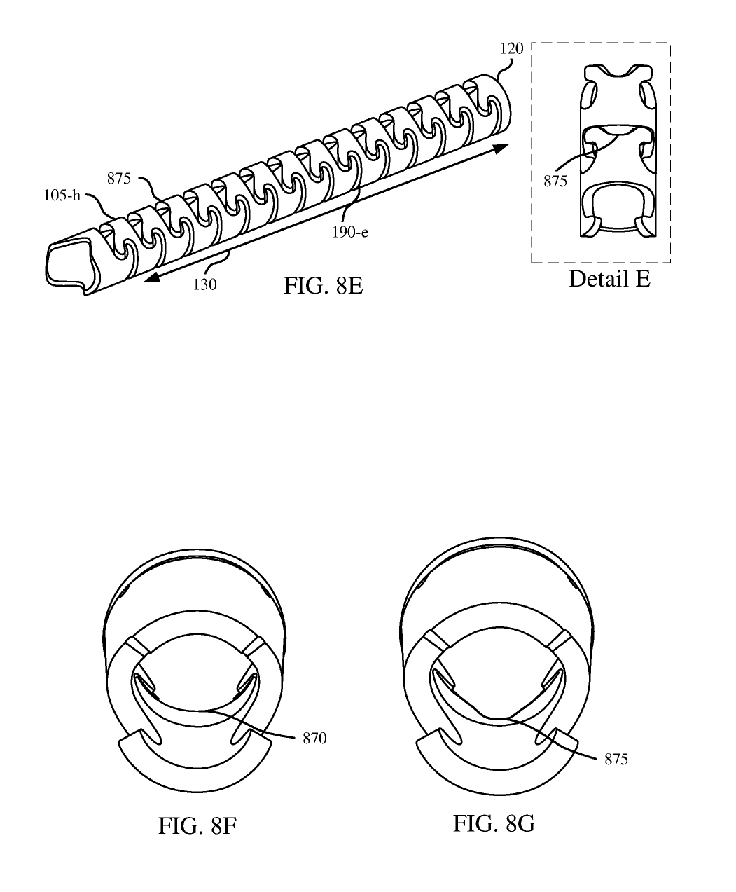

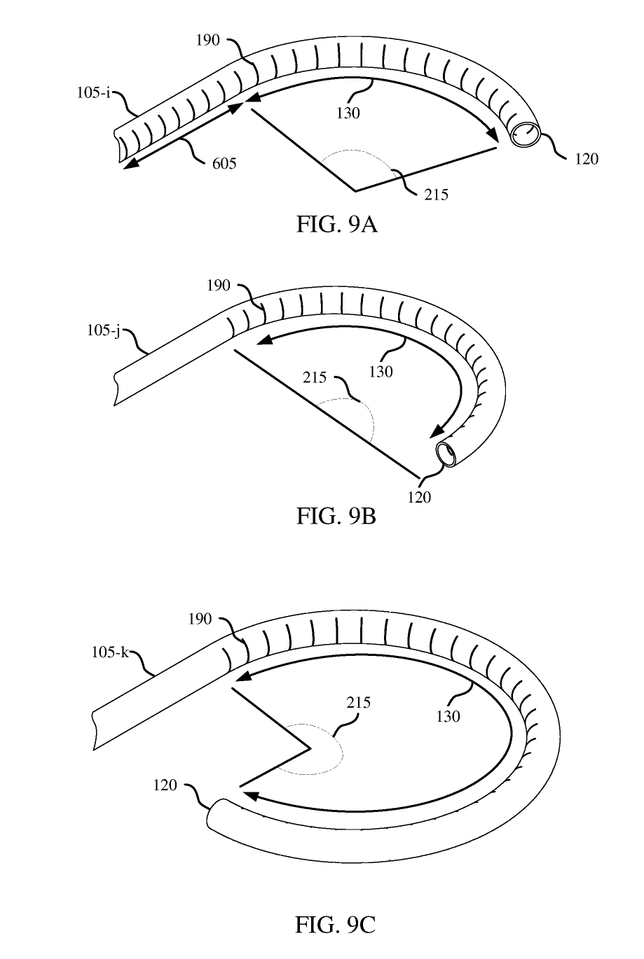

In accordance with various embodiments, the anchoring force exhibited by the nonlinear distal section 130 (i.e., the amount of force required to pull the cannula 105-c back through the pierced access hole by at least partially straightening out the nonlinear distal section 130) may vary depending on several characteristics of the cannula such as material properties, the size (e.g., outer and inner diameter) of the cannula, the number, shape and size of apertures 190 along the distal section 130 (as described in more detail with reference to FIGS. 8A-8D), and the angle of sweep 215 of the nonlinear shape (as described in more detail with reference to FIGS. 9A-9C).

According to aspects of the present disclosure, the anchoring force exhibited by different cannula configurations may be quantified experimentally. For example, a cannula may be pierced through a test material (e.g., silicon) and then allowed to transition into the nonlinear configuration. Then, the cannula may be pulled back through the test material with an apparatus that measures the pulling force (e.g., Instron load cell) until the nonlinear portion of the cannula has straightened out and completely passed back through the pierced hole. In this way, the force required to pull the cannula back through the test material (e.g., the average force or maximum force) may be quantified for different cannula configurations. In accordance with various embodiments described herein, the anchoring force exhibited by various cannula configurations tested in the fashion may range from approximately 0.15 lbf to approximately 0.50 lbf.

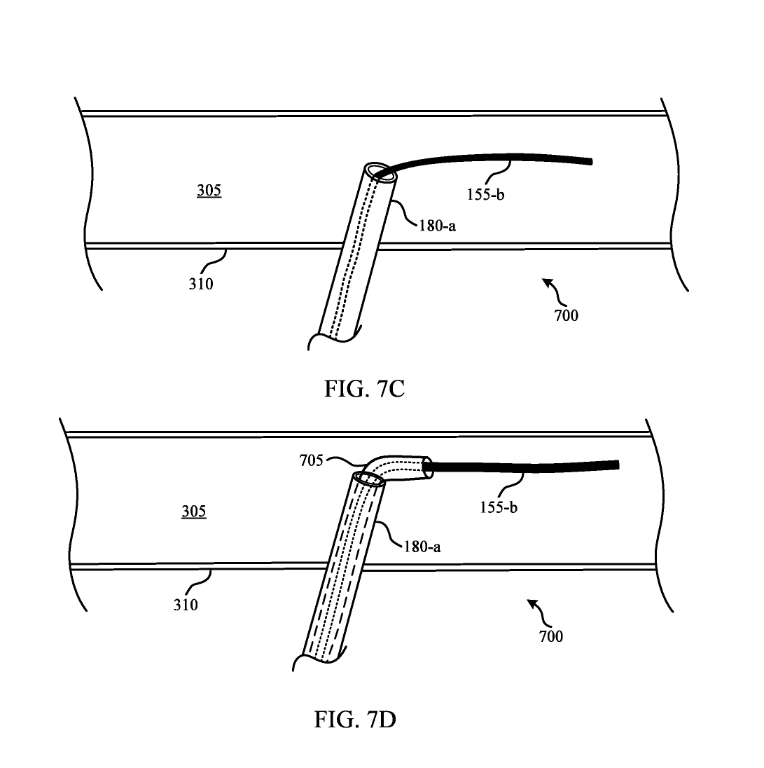

With reference to FIGS. 6A-6B, a schematic view of a system 600 for providing access to a body lumen 305 is illustrated in accordance with various embodiments. The system 600 includes a guide wire 155-b slidably disposed within a cannula 105-d, which may be examples of the cannula 105 and the guide wire 155 described with reference to any of the FIGS. 1-5. The cannula 105-d illustrated in FIG. 6A includes a distal section 130 that is already transitioned into a nonlinear shape. As shown, the angle of sweep 215 of the nonlinear shape exceeds 90°, and is approximately 130°. Distal section 130 includes a plurality of apertures 190 that impart additional flexibility to distal section 130 as described with reference to various embodiments. The apertures 190 may also be disposed along a section 605 that is proximal to distal section 130. Section 605 is a portion of cannula 105-d that is not configured to passively transition into a nonlinear configuration (when for example, an internal stylet 145 is withdrawn), but because of apertures 190, is more flexible than more proximal portions of the cannula 105-d that are free from the apertures 190. Because of this increased flexibility of section 605, the cannula 105-d may be advanced further into the body lumen 305 over the guide wire 155-b, as illustrated in FIG. 6B. The ability to advance the cannula 105-d deeper into the body lumen 305 may be advantageous in circumstances where placing the distal end 120 of the cannula 105-d closer to the obstruction or target area is desired.

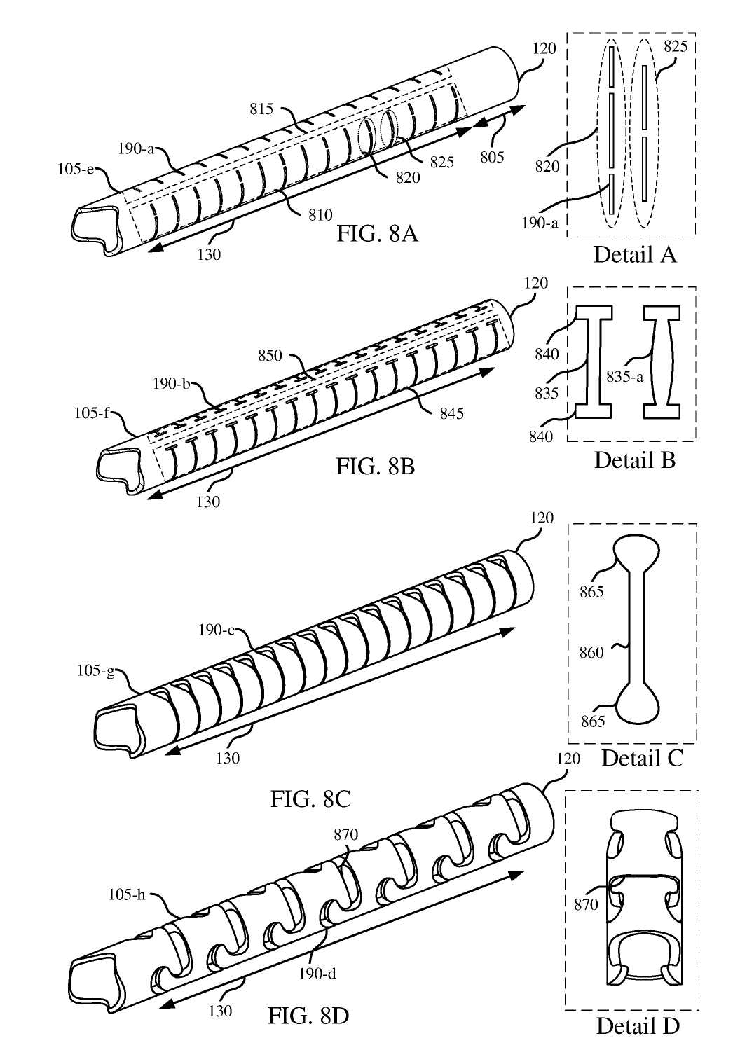

With reference to FIG. 7A, a schematic view of a system 700 for providing access to a body lumen 305 is illustrated in accordance with various embodiments. The system 700 includes a guide wire 155-b slidably disposed within a cannula 105-b, which is slidably disposed within a sheath 180-a. The guide wire 155-b, cannula 105-b, and sheath 180-a may be examples of the guide wire 155, cannula 105, and sheath 180 described with reference to any of the FIGS. 1-6. As illustrated in FIG. 7A, the sheath 180-a may remain outside of the lumen 305 while the distal section 130 of the cannula 105-b pierces through the lumen wall 310. However, as illustrated in FIG. 7B, the sheath 180-a may also be advanced over the cannula 105-b, through the lumen wall 310, and into the body lumen 305, thereby providing a continuous conduit from the proximal handle assembly 170 to the body lumen 305. In such cases, the sheath 180-a may be, or may serve as, a stent delivery catheter through which a stent is delivered into the body lumen 305. Alternatively, instead of advancing the sheath 180-a into the body lumen 305, the cannula 105-b and the sheath 180-a may be withdrawn, leaving the guide wire 155-b within the body lumen 305, and a totally different sheath (e.g., a separate stent delivery catheter) may be advanced over the guide wire 155-b and into the body lumen 305.

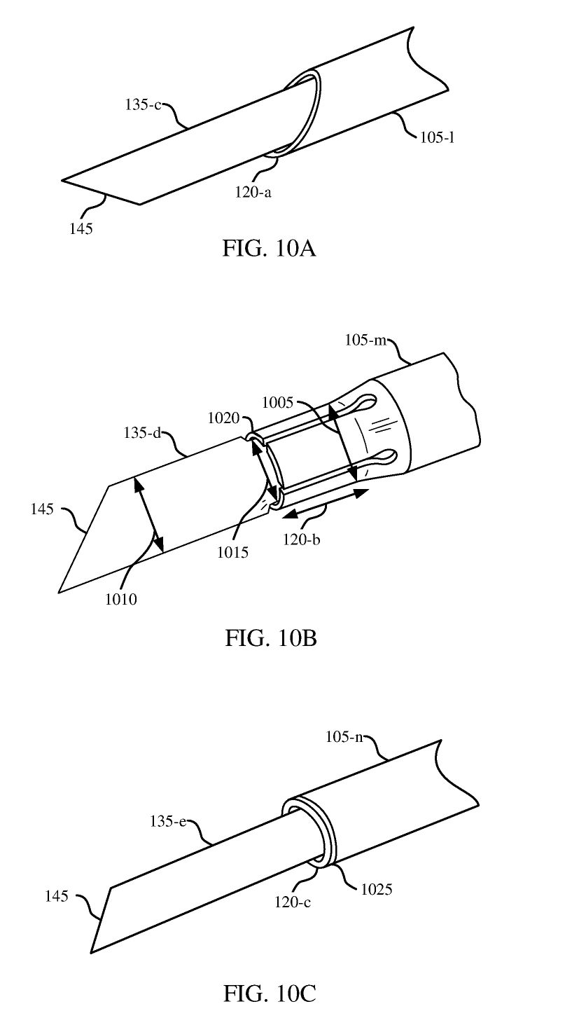

With reference to FIG. 7C, after advancing the sheath 180-a over the cannula 105-b into the body lumen 305, the cannula 105-b may be withdrawn, and the sheath 180-a may remain as a conduit for delivery of another device over the guide wire 155-b into the body lumen 305. With reference to FIG. 7D, in some examples, with the sheath 180-a within the body lumen (after being advanced over a cannula 105-b and/or a guide wire 155-b), a stent delivery catheter 705 may be advanced through the sheath 180-a and into the body lumen 305. The sheath 180-a may include features on the distal end that facilitate the advancement through the lumen wall 310. For example, the distal end of the sheath 180-a may be tapered or may include an energy delivery element. Examples of suitable energy delivery elements, such as energy-based cutting elements, are described below with reference to FIG. 10C.

With reference to FIGS. 8A-8E, various embodiments of a cannula 105 are illustrated with a plurality of apertures 190 disposed along a length of the distal section 130. In general, the apertures 190 impart flexibility to the distal section 130, thereby allowing the distal section 130 to transition into a nonlinear shape as described with reference to various embodiments of the present disclosure. In certain embodiments, the apertures 190 extend through the entire thickness of the wall of the cannula 105. Alternatively, some or all of the apertures 190 may only partially penetrate through the wall of the cannula 105 (i.e., a notch).

The length of the distal section 130 containing apertures 190 may vary in different embodiments, and as described below, may affect the size and shape of the nonlinear shape. In accordance with various embodiments, the distal section 130 containing apertures 190 may range from approximately 0.20 inches to approximately 1.0 inches. Also, as shown in FIG. 8A, in some embodiments, there is a portion 805 of the cannula 105-e distal to the aperture pattern that is free from apertures 190. Such a feature may provide additional rigidity near the distal end 120, thereby helping to coaxially align an internal member (e.g., a stylet 135) as it exits the distal tip 120, which may result in a reduced pierce force, as described with reference to FIG. 3. In some examples, the length of the portion 805 is between approximately 0.05 and 0.20 inches. Alternatively, the apertures 190 may extend all the way to the distal end 120, as illustrated in FIG. 8B. Also, although the apertures 190 are illustrated as extending only along the distal section 130, it may be appreciated that the apertures 190 may extend proximal to the distal section 130, as described with reference to FIGS. 6A-6B.

In accordance with the present disclosure, the size and shape of the apertures 190, the spacing of the apertures 190, and the overall length of the pattern of apertures 190 may be optimized to yield certain characteristics that may be advantageous for luminal access and guide wire placement. For example, the apertures 190 may be uniform in size, shape and spacing, or may be varied as desired to optimize flexibility, radius of curvature, angle of sweep, strength, fatigue resistance, etc. In additional to varying the pattern of apertures 190, the wall thickness of the cannula 105 may also be varied over the length of the distal section 130 for variable flexibility profiles, increased strength near stress risers, or the like. For example, the thickness of the wall of the cannula 105 at the proximal end of the distal section 130 may be thicker than wall at the distal end 120 of the cannula 105.

It may be appreciated that the presence of apertures 190 in the wall of the cannula 105 create inherent stress risers that weakens the cannula wall, making it susceptible to fracture under extreme strain or flexural fatigue. As such, the wall thickness of the cannula 105 should be chosen in concert with the aperture design and pattern to achieve an appropriate resistance to fracture. Also, the shape of the apertures 190, the length and width of the apertures 190, and the spacing of the apertures 190, are all variables that can be optimized for the desired performance or medical procedure. For example, the proximal-most aperture 190 may have a different design than the rest of the apertures 190, in order to be more resistant to fracture due to this being a significant stress riser feature. In some examples, instead of a single proximal-most aperture 190, there may be a proximal group of apertures 190 that differ in size or shape from a distal group of apertures 190, which may reduce the stress riser profile caused by the distal group of apertures 190.

The apertures 190 may be formed with a laser cutting process; however, other machining processes may be used such as milling, etching, electro-polishing, and electrical discharge machining (EDM). The apertures 190 may be formed while the cannula 105 is straightened out (i.e., before heat setting the distal section 130).

Turning to FIG. 8A, the distal section 130 of a cannula 105-e is illustrated with a plurality of rectangular apertures 190-a (i.e., slits) in accordance with various embodiments. As illustrated, the rectangular apertures 190-a may be oriented substantially orthogonal to a longitudinal axis defined by the lumen 110 of the cannula 105-e. The rectangular apertures 190-a may be uniformly spaced along the distal section 130 and may all have equal widths (i.e., the smaller of the two rectangular dimensions) and lengths. Alternatively, the size, orientation, and the spacing between the rectangular apertures 190-a may be varied to impart a variable stiffness or shape profile to the distal section 130 or to reduce the magnitude of stress risers caused by the rectangular apertures 190-a. In a particular embodiment, the width of the rectangular apertures 190-a is approximately 0.0010 inches and the spacing between the rectangular apertures 190-a is approximately 0.0050 inches. By making the rectangular apertures 190-a relatively thin and densely spaced (in relation to the dogbone apertures 190-b described in FIG. 8B, for example), the nonlinear shape of the distal section 130 may deflect with greater uniformity (i.e., a smoother bend). Also, dense spacing may reduce the magnitude of stress risers that could cause weakening and fracture of the distal section 130.

In a particular embodiment, the rectangular apertures 190-a are arranged into groups that form two rows that are located on diametrically opposed sides of the cannula 105-e. For example, as shown, in FIG. 8A, the rectangular apertures 190-a may be grouped into a first row 810 and a second row 815 that is diametrically opposite the first row 810. The rectangular apertures 190-a in each of the two rows 810, 815 may be generally symmetric (e.g., same size, orientation, and spacing) or the sizing and spacing may be different between the two rows, thereby imparting a flexural bias into the distal section 130 (i.e., the distal section 130 bends more easily in one direction than the other). However, in some embodiments, the cannula 105-e may only include a single row of rectangular apertures 190-a (e.g., row 810), which may correspond to the internal radius of the nonlinear shape when curved.

Moreover, the rectangular apertures 190-a within each row 810, 815 may be further arranged into a plurality of groups, such as groups 820, 825 for example (shown more clearly in Detail A). Each of the groups may comprise two or more linearly-aligned rectangular apertures 190-a (i.e., aligned end-to-end). For example, group 820 contains three linearly-aligned rectangular apertures 190-a (e.g., short-long-short) and group 825 contains two linearly-aligned rectangular apertures 190-a (e.g., each of the same length). Detail A shows a schematic view of rectangular apertures 190-a of the groups 820, 825 if laid out flat. Staggering the gaps between linearly-aligned rectangular apertures 190-a may reduce the magnitude of stress risers throughout the distal section 130.

In accordance with various embodiments, the length (i.e., the longer of the two rectangular dimensions) of the rectangular apertures 190-a within the various groups may be sized such that the edges of the groups align along a common circumferential location on the cannula 105-e (as shown in FIG. 8A). Alternatively, the edges of the groups may instead be circumferentially offset from any adjacent group (as shown in Detail A). Staggering the circumferential location where the rectangular apertures 190-a of each group terminate may also help reduce the magnitude of stress risers throughout the distal section 130. Furthermore, some of the groups may contain rectangular apertures 190-a that have a width that is greater than the width of the rectangular apertures contained in other groups. For example, the rectangular apertures 190-a in the distal-most or proximal-most groups may be sized differently than the other internal groups, because the stress risers are the greatest at these proximal and distal locations.

With reference to FIG. 8B, the distal section 130 of a cannula 105-f is illustrated with a plurality of dogbone apertures 190-b in accordance with various embodiments. The dogbone apertures 190-b generally include a central portion 835 and two end portions 840 that are aligned substantially orthogonal to the central portion 835 (see Detail B). In accordance with various embodiments, the central portion 835 is rectangular in shape. Alternatively, the central portion 835-a may be elliptical in shape. As shown, the dogbone apertures 190-b may be aligned along the distal section 130 such that the central portions 835 are substantially orthogonal to a longitudinal axis defined by the lumen 110 of the cannula 105-f.

Similar to the rectangular apertures 190-a described above, the dogbone apertures 190-b may be arranged into two rows that are located on diametrically opposite sides of the cannula 105-f, such as row 845 and row 850. In some embodiments, the dogbone apertures 190-b in each of the two rows 845, 850 are generally symmetric (e.g., same width/shape of central portion 835 and same spacing). In other embodiments, the central portion 835 of the dogbone apertures in row 845 may have a different width or shape than those of row 850, thereby imparting a flexural bias into the distal section 130 (i.e., the distal section 130 bends more easily in one direction than the other). However, in some embodiments, the cannula 105-f may only include a single row of dogbone apertures 190-b (e.g., row 845), which may correspond to the internal radius of the nonlinear shape when curved.

Because the inner curve of the nonlinear shape causes the cannula wall along the distal section 130 to compress, and the outer curve causes the wall to elongate, the dogbone apertures 190-b may be configured to accommodate the compression and extension of the cannula wall material. For example, the dogbone apertures 190-b for the inner radius (i.e., the inside of the curved shape) may include one or more wider features (e.g., elliptical cross section 835-a) than the apertures on the outer radius, so that as the cannula wall along the inner radius compresses, the thicker elliptical cross section 835-a will provide sufficient space for the wall material to come together before the opposing edges of the aperture touch. Otherwise, the opposing edges of the apertures may come into contact prematurely, thereby limiting the flexibility of the distal section 130.

The spacing between dogbone apertures 190-b may be uniform or may instead be varied to impart variable flexibility along the distal section 130. For example, a particular embodiment includes dogbone apertures 190-b that are uniformly spaced along the distal section 130 of the cannula 105-f at a spacing of approximately 0.020 inches, with rectangular-shaped central portions 835 with a thickness of approximately 0.0010 inches and elliptical-shaped central portions 835-a with a thickness of approximately 0.0030 inches.

With reference to FIG. 8C, the distal section 130 of a cannula 105-g is illustrated with a plurality of curved apertures 190-c in accordance with various embodiments. The curved apertures 190-c may include a central portion 860 that is generally rectangular, and two rounded-shaped ends 865, as shown in Detail C. It may be appreciated that the rounded-shaped ends 865 generally create less of a localized stress riser than rectangular corners, and thereby may increase the fatigue strength of the distal section 130.

Moreover, the curved apertures 190-c may be disposed along one side of the cannula 105-g only (as opposed to two diametrically opposite rows of apertures). In such embodiments, the curved apertures 190-c may be arranged along the inner radius of the nonlinear shape when curved. Arranging the curved apertures 190-c in this way provides the needed space for material compression along the internal radius (as described with reference to FIG. 8B). Additionally, such an aperture arrangement provides for a smooth internal surface along the outer radius of the lumen 110 of the nonlinear shaped distal section 130 of the cannula 105-g, which may facilitate the distal advancement of an internal member (e.g., a guide wire 155) without getting caught by the aperture features and causing frictional drag on the guide wire 155, thereby impeding smooth movement. It should be appreciated that any of the apertures 190 described herein may be disposed only along the internal curved surface of the distal section 130 to reduce catching of an internal guide wire 155 during advancement.

With reference to FIG. 8D, the distal section 130 of a cannula 105-h is illustrated with a plurality of S-cut apertures 190-d in accordance with various embodiments Similar to the curved apertures 190-c described with reference to FIG. 8C, the S-cut apertures 190-d may be disposed only along one side of the cannula 105-h (e.g., along the internal radius of the nonlinear shape when curved). The S-cut apertures 190-d may generally be characterized as including one or more curved features (e.g., S-shapes, elbows, or non-uniform curved features) on substantially diametrically opposite sides of the cannula 105-h that are connected by a generally straight cut. The S-cut apertures 190-d may form linkage-like connections along the distal section 130 of the cannula 105-h. Detail D shows a top view of the S-cut apertures 190-d and a lip feature 870 formed by the substantially straight cut connecting the two S-shaped cuts on either side of the cannula 105-h. As described in more detail below, as the cannula 105-h bends, the lip feature 870 will pivot and protrude inwards towards the center of the cannula lumen, thereby creating a series of ledge features along the internal lumen.

With reference to FIG. 8E, the distal section 130 of another example of a cannula 105-h is illustrated with a plurality of scalloped S-cut apertures 190-e in accordance with various embodiments. Scalloped S-cut apertures 190-e are similar to S-cut apertures 190-d illustrated with referenced to FIG. 8D, except that scalloped S-cut apertures 190-e include a cut-out feature 875 along the central cut that connects the two S-shaped cuts on the opposite sides of the cannula 105-h. Detail E shows a top view of cannula 105-h and more clearly illustrates the cut-out feature 875, which may be characterized as a V-notch, parabolic shape, or the like. As described in more detail below, the cut-out feature 875 may reduce the size of the lip or ledge feature that protrudes into the cannula lumen when the cannula 105-h bends.

One or more features of the S-cut apertures 190-d or 190-e may be designed to reduce the tendency of an internal member (e.g., stylet 135 or guide wire 155) to become caught on or otherwise impeded by one of the apertures as the internal member is advanced distally or withdrawn proximally through the cannula 105-h. As described above, the S-cut apertures 190-d or 190-e may be disposed along the distal section 130 of the cannula 105-h such that when the distal section 130 is curved (e.g., when the distal section 130 takes its pre-shaped configuration), the S-cut apertures 190-d or 190-e are disposed along the internal radius of the curved section. In such examples, when an internal member is advanced distally through the lumen of the cannula 105-h, the internal member may tend to slide along the outer radius of the curved section, which may be free from any apertures. As such, the internal member may glide through the lumen distally without getting caught between the voids caused by the S-cut apertures 190-d or 190-e.

However, in some cases, as the internal member is withdrawn proximally through the lumen of the cannula 105-h, the internal member may get caught on or otherwise impeded by one or more of the internal lips or ledges that are created by the S-cut apertures 190-d or 190-e when the cannula 105-h is curved. FIG. 8F illustrates a cross sectional view of the cannula 105-h with S-cut apertures 190-d as the cannula 105-h is in a curved configuration. As shown in FIG. 8F, the lip feature 870 protrudes inwards into the lumen of the cannula 105-h. In some cases, these lip features 870 may impede the proximal withdrawal of an internal member because the internal member may drag along the edges of the lip features 870.

FIG. 8G shows a cross sectional view of the cannula 105-h with scalloped S-cut features 190-e as the cannula 105-h is in a curved configuration. As illustrated in FIG. 8G, the cut-out feature 875 may reduce the amount of material (e.g., the size of the lip) that would otherwise have protruded internally to create a ledge feature. As such, the scalloped S-cut apertures 190-e may reduce the drag experienced by an internal member as it is withdrawn proximally through the cannula 105-h.

Other features or designs of the S-cut apertures 190-d or 190-e may be modified to further reduce the impedance of movement experienced by an internal member as it is distally advanced or proximally withdrawn through the cannula 105-h. For example, the overall dimensions of the S-cut apertures 190-d or 190-e may be reduced, which would reduce the size of the lip that is created when the cannula 105-h is bent. Additionally or alternatively, the spacing between adjacent S-cut apertures 190-d or 190-e may be reduced, which may create a more uniform, closely spaced, series of ledges along the internal lumen of the cannula 105-h, which may in turn reduce the frictional force experienced by an internal member is it drags over the ledges (due to the distribution of force over many closely-spaced ledges as compared with more spaced-apart ledges). Also, in some examples, the S-cut apertures 190-d or 190-e may be oriented (e.g., with respect to the distal end 120 of the cannula 105-h) such that when the cannula 105-h is bent, the series of internal ledges are stepped in such a way that each subsequent ledge is lower than the adjacent ledge (from the perspective of an internal member moving through the cannula 105-h in the proximal direction). In other words, instead of an internal member being dragged up and over each ledge (as the internal member is being proximally withdrawn), the internal member is being dragged over and down each ledge.

It may be appreciated that the aperture shapes, sizes, and patterns illustrated with reference to FIGS. 8A-8E are exemplary and that other aperture shapes, sizes, or patterns may be considered without departing from the scope of the present disclosure. As described above, certain aperture shapes, patterns, features, and arrangements may exhibit relative advantages regarding flexibility, strength, resistance to fatigue fracture, torqueability, and prevention of catching of an internal member (e.g., stylet 135 or a guide wire 155). Accordingly, one or more of the aperture shapes or patterns described above may be combined in various ways to yield a combination pattern with the combined advantages of each of the constituent aperture patterns in accordance with the described embodiments. For example, a cannula 105 may include a particular aperture shape (e.g., rectangular apertures 190-a) along a more proximal portion of distal section 130 and then a different aperture shape (e.g., dogbone apertures 190-b or S-cut apertures 190-d) along a more distal portion of distal section 130 to impart a varied flexural profile or to selectively provide additional strength at areas with high stress risers. Alternatively, a cannula 105 may include a particular aperture shape along the inner radius of the nonlinear shape and a different aperture shape along the outer radius. It should be noted that the concept of confining the apertures to the internal radius of the distal section 130 to avoid impeding guide wire movement is not limited to any particular aperture design.

In some embodiments, a thin polymeric overjacket (e.g., polytetrafluoroethylene (PTFE), polyethylene terephthalate (PET), silicone, or the like) may be placed over the distal section 130 (e.g., through heat shrinking, casting, dipping, or spraying) to cover some or all of the apertures 190. Such an overjacket may facilitate the advancement of fluid (e.g., aspiration of body fluids or injection of contrast) through the cannula 105 by sealing off the apertures 190. In addition, an overjacket may reduce the friction forces between the outer diameter of the cannula 105 and the inner diameter of the channel through which it travels (e.g., working channel of an endoscope or a sheath 180), which may advantageously reduce the force needed to advance the cannula 105 through the body. Moreover, if the distal section 130 of the cannula 105 were to break while inside a patient (e.g., inside a lumen or within the endoscope), the overjacket may retain the pieces of the cannula 105 together, thereby preventing pieces from being lost within the patient. The overjacket can also be extended beyond the distal end 120 of the cannula 105 to form a tip that is less traumatic and less likely to bind a guide wire 155 passing through it. The polymeric overjacket may only cover the distal section 130 of the cannula 105, or may extend about 1 mm or more or less.

Turning to FIGS. 9A-9C, various embodiments of a cannula 105 are illustrated with the distal section 130 transitioned to a nonlinear shape of various angles of sweep 215 in accordance with various embodiments. It may be appreciated that the particular embodiments described herein are examples and that a variety of angles of sweep 215 other than those illustrated may be employed without departing from the scope of the present disclosure. For example, in accordance with various embodiments, the angle of sweep 215 of the distal section 130 may range from 0° to 480°.

As described with reference to FIGS. 8A-8E, the angle of sweep 215 of the distal section 130 may be adjusted by adjusting the length of the distal section 130 that includes apertures 190. In general, the longer the length of the distal section 130 containing apertures 190, the greater the angle of sweep 215 will be. In a similar manner, the radius of curvature 210 may be adjusted by varying the width or spacing of the apertures 190. For example, a tighter radius of curvature 210 can be formed with wider apertures 190 or denser spacing between adjacent apertures 190. In contrast, a larger radius of curvature 210 can be formed with thinner apertures 190 or wider spacing between adjacent apertures 190.

With reference to FIG. 9A, the distal section 130 of a cannula 105-i is illustrated with an angle of sweep 215 of approximately 135°. Such an embodiment may provide a good balance between retention properties (i.e., anchoring within the body lumen) and direction control. As described with reference to FIGS. 5A-5B, the distal section 130 may be inserted into a body lumen and the angle of sweep 215 may be reduced by deploying a guide wire 155 until the distal end 120 is oriented in a preferred direction. In a particular embodiment, a length of the distal section 130 containing apertures 190 of approximately 0.40 inches will yield an angle of sweep 215 of approximately 135°.

As shown, in some embodiments, the cannula 105-i may include apertures 190 along a section 605 that is proximal to the distal section 130. The section 605 may be a portion of the cannula 105-i that is not pre-shaped (e.g., by heat setting), but that still includes one or more apertures 190. Accordingly, section 605 is more flexible than portions of the cannula 105-i without apertures 190, but will remain substantially straight in the absence of deflection forces. Although the section 605 is illustrated as including rectangular apertures 190, it should be appreciated that section 605 may include any aperture 190 or aperture pattern described herein and may be the same or different from the apertures 190 disposed along distal section 130. In certain aspects, the section 605 is flexible enough to track over a guide wire 155 into a body lumen (e.g., common bile duct 605), as illustrated with reference to FIGS. 6A-6B.

With reference to FIG. 9B, the distal section 130 of a cannula 105-j is illustrated with an angle of sweep 215 of approximately 180°. Such an embodiment may provide additional retention force over the cannula 105-i because the distal end 120 turns back and abuts relatively orthogonally against the lumen wall, opposing the tendency of the cannula 105-j to slip backward and fall out of the lumen. However, as described with reference to FIGS. 5A-5B, the clinician may need to reduce the angle of sweep 215 (i.e., straighten out the distal section 130) to about 90° (or some other desired angle depending on the pierce angle 315) to be able to advance a guide wire 155 in a preferred direction. In a particular embodiment, a length of the distal section 130 containing apertures 190 approximately 0.60 inches yields and angle of sweep 215 of approximately 180°

With reference to FIG. 9C, the distal section 130 of a cannula 105-k is illustrated with an angle of sweep 215 of approximately 270°. In such an embodiment, the distal section 130 provides a relatively atraumatic anchoring feature, by virtue of the fact that a curved portion of the distal section 130 abuts against the lumen wall (as opposed to the distal end 120). With this embodiment, the distal section 130 of the cannula 105-k creates a cloverleaf path for the guide wire 155 as it passes into the target lumen. However, as with previously described embodiments, the angle of sweep 215 can be adjusted with a guide wire 155, such that as the guide wire 155 is advanced through the distal end 120, the distal section 130 straightens out allowing direction-controlled placement of the guide wire 155. In a particular embodiment, a length of the distal section 130 containing apertures 190 of approximately 0.80 inches yields an angle of sweep 215 of approximately 270°.

Alternatively, instead of straightening out the distal section 130, in some embodiments, the distal section 130 can remain in the approximately 270° cloverleaf shape, and the guide wire 155 would still exit along a longitudinal path through the lumen (see FIG. 5A). If the orientation of the distal end 120 is in an undesired direction (e.g., retrograde), the distal end 120 could be rotated 180 degrees (as described with reference to FIGS. 4A-4B), so that the guide wire 155 is directed along the desired direction (e.g., antegrade) within the lumen. The guide wire 155 could then be advanced from the distal end 120 without needing to straighten out the distal section 130 (e.g., by using a guide wire 155 without a relatively stiff portion 165, as described with reference to FIG. 5B).

As illustrated, cannula 105-k includes apertures 190 disposed along the distal section 130. It should be appreciated that any aperture type (e.g., rectangular apertures 190-a, dogbone apertures 190-b, curved apertures 190-c, S-cut apertures 190-d, or scalloped S-cut apertures 190-e) or aperture pattern described herein may be used to create a distal section 130 with an angle of sweep 215 or approximately 270°. Another feature illustrated by FIG. 9C is that in accordance with certain aspects, the apertures 190 may be disposed along the distal section 130 such that there are no aperture features along the portion of the cannula 105-k defining the outer curved surface of the distal section 130. Accordingly, if an internal member (e.g., a guide wire 155 or stylet 145) is advanced through the lumen of the cannula 105-k while the distal section 130 is curved, there may be less chance of the internal member from getting caught in an aperture 190 because of the tendency of the internal member to hug the outer curve of the distal section 130.

With reference to FIGS. 10A-10C, various embodiments of the distal end 120 of the cannula 105 are described. In some embodiments, the distal end 120 of the cannula 105 may be blunt and generally tubular and uniform in shape. Alternatively, the distal end 120 may be tapered, beveled, or include one or more internal or external alignment features that generally assist in the piercing of tissue by reducing the force needed to access a body lumen. With reference to FIG. 10A a stylet 135-c is shown protruding from the distal end 120-a of a cannula 105-1. The stylet 135-c and cannula 105-1 may be examples of any stylet 135 and cannula 105 described with reference to any of the preceding figures. As shown, the distal end 120-a is beveled (e.g., at a 45° angle) in certain embodiments. Although the distal end 120-a may be beveled, the edges of the cut surface may be polished or otherwise softened so as to not be sharp, thereby reducing the potential to cause trauma to the lumen wall after piercing. Alternatively, the beveled distal end 120-a may be sharpened or otherwise configured to pierce tissue, such as the duodenal wall. When sharpened, the beveled distal end 120-a may include multiple bevels at various angles to optimize the piercing characteristics of the distal end 120-a.

In certain aspects, in order to further improve the ability of a cannula 105 to follow behind a stylet 135 to pierce through tissue, the cannula 105 may include one or more features to reduce the profile of the cannula 105 near its distal end 120 or to coaxially align the two members as the cannula 105 and stylet 135 pierce tissue. Turning to FIG. 10B, a cannula 105-m with a tapered distal end 120-b is illustrated in accordance with various embodiments. As shown, the tapered distal end 120-b reduces the outer and inner diameter of the cannula 105-m along the distal end 120-b. The reduced outer diameter 1005 of the cannula 105-m along the distal end 120-b may match closely to the outer diameter 1010 of the stylet 135-d near its distal end 145, thereby reducing or eliminating any lateral gap or offset. As such, the diametrical profile from the stylet 135-d to the cannula 105-m gradually and smoothly increases, thereby reducing the force required to advance the cannula 105-m through tissue behind the stylet 135-d.

To facilitate the reduced inner diameter of the cannula 105-m along the distal end 120-b, the stylet 135-d may include a portion with a reduced diameter 1015. The reduced diameter 1015 of the stylet 135-d may be formed by necking, drawing, or grinding down the outer diameter of the stylet 135-d. In some embodiments, the reduced diameter 1015 extends only along a length of the stylet 135-d to accommodate the reduced diameter 1005 of the distal end 120-b of the cannula 105-m, thereby forming an hour glass cross sectional profile of the stylet 135-d. In other examples, the entire stylet 135-d proximal to the distal end with diameter 1010 may have a reduced diameter 1015.

As illustrated, the distal end 120-b of the cannula 105-m may include a plurality of tabs or flaps 1020, which may be configured to flare open, bulge, or otherwise flex to accommodate the withdrawal of the portion of the stylet 135-d with diameter 1010. Once the portion of the stylet 135-d with diameter 1010 is fully withdrawn into the cannula lumen 110, the flaps 1020 would return to the reduced diameter 1005.

With reference to FIG. 10C, a cannula 105-n with an energizable distal end 120-c is illustrated in accordance with various embodiments. The energizable distal end 120-c may include an energy-based cutting element 1025 configured to cut or ablate tissue with the energy (e.g., radiofrequency energy). For example, the energy-based cutting element 1025 may include a diathermic or dielectric cutting element including but not limited to a dielectric cautery ring, a cutting knife, a cutting wire, pinching cutters, or the like.

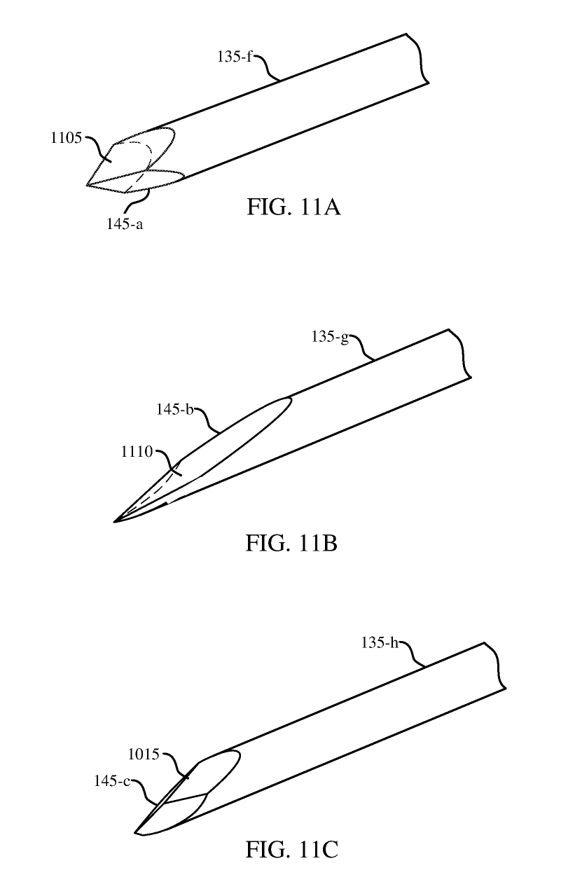

With reference to FIGS. 11A-11C, various embodiments of the distal tip 145 of the stylet 135 are illustrated. Any of the distal tips 145 may be used in combination with any of the stylets 135 described herein, and any combination of distal tip 145 and stylet 135 may be used with any cannula 105 described herein and incorporated into any access system described herein. With reference to FIG. 11A, a trocar grind distal end 145-a is illustrated. The trocar distal end 145-a may be characterized by three bevels 1005 which are disposed equidistant (i.e., at 120° intervals) around the circumference of the cannula 135-f. These three bevels 1105 may be sloped at an angle of approximately 15° with respect to the longitudinal axis of the stylet 135-f and may terminate at a point, but it should be appreciated that the angle may be varied to increase or decrease the sharpness of the distal end 145-a. Turning to FIG. 11B, a hypodermic grind distal end 145-b of a stylet 135-g is illustrated. The hypodermic distal end 145-b may be characterized by a single bevel 1110. The angle of the single bevel 1110 with respect to the longitudinal axis of the stylet 135-g may vary, but is approximately 15° in some examples. With reference to FIG. 11C a four-plane grind distal end 145-c is illustrated, and may be characterized by three bevels 1015 as shown. In the four-plane grind distal end 145-c, the proximal-most bevel may be at a first angle with respect to the longitudinal axis of the stylet 135-h (e.g., 15°) and the other two side bevels may be at a greater angle with respect to the longitudinal axis of the stylet 135-h (e.g., 30°).

In certain situations the clinician may wish to re-straighten out the distal end 130 of a cannula 105 after it has already been transitioned into a nonlinear shape. For example, the clinician may have inadvertently pierced the wrong body lumen, the cannula 105 may have fallen out of the body lumen, or the clinician may wish to re-straighten the distal section 130 for some other reason. In any case, re-advancing the internal stylet 135 distally (which at this point has already been proximally retracted at least past the distal section 130) may be difficult because the stylet 135 may catch one or more of the apertures disposed along the distal section 130. Therefore, in accordance with certain aspects of the disclosure, a clinician may retract the cannula 105 back into an outer sheath 180 to re-straighten the distal section 130 of the cannula to a substantially linear configuration.

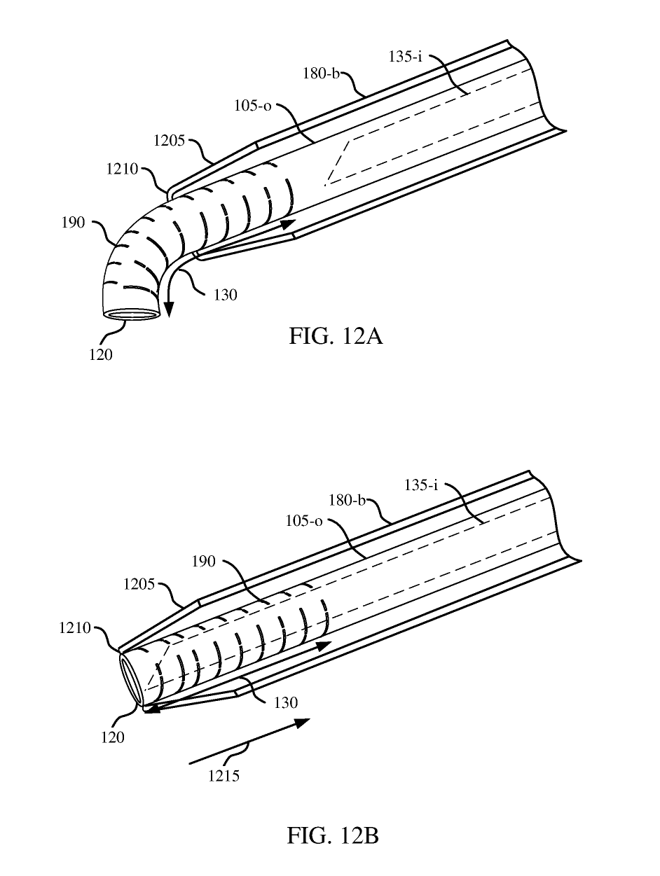

For example, with reference to FIGS. 12A-12B an outer sheath 180-b (shown in a cross-sectional view), a cannula 105-o, and a stylet 135-i (shown in phantom lines) are illustrated in accordance with various embodiments of the present disclosure. The sheath 180-b, cannula 105-o, and stylet 135-i may be an example of a sheath 180, cannula 105, and stylet 135 described with reference to any of the preceding figures. In FIG. 12A, the sheath 180-b includes a tapered portion 1205 that functions to reduce the diameter of the sheath 180-b at the distal end 1210. Accordingly, the gap between the outer diameter of the cannula 105-o and the inner diameter of the sheath 180-b is reduced or eliminated at the distal end 1210. As such, in accordance with various embodiments, the sheath 180-b may be used to re-straighten out the distal section 130 of the cannula 105-o by sliding the sheath 180-b distally with respect to the cannula 105-o, or by withdrawing the cannula 105-o into the sheath 180-b.

For example, a clinician may retract the cannula 105-o in a proximal direction 1215, thereby causing the distal section 130 of the cannula 105-o to straighten out as it passes the distal end 1210 of the sheath 180-b. The same result may be achieved by advancing the sheath 180-b distally with respect to the cannula 105-o. In either case, once the distal section 130 of the cannula 105-o has been straightened out, the internal stylet 135-i may then be re-advanced distally through the distal section 130 without catching any of the apertures 190, and the procedure of accessing a body lumen may resume.

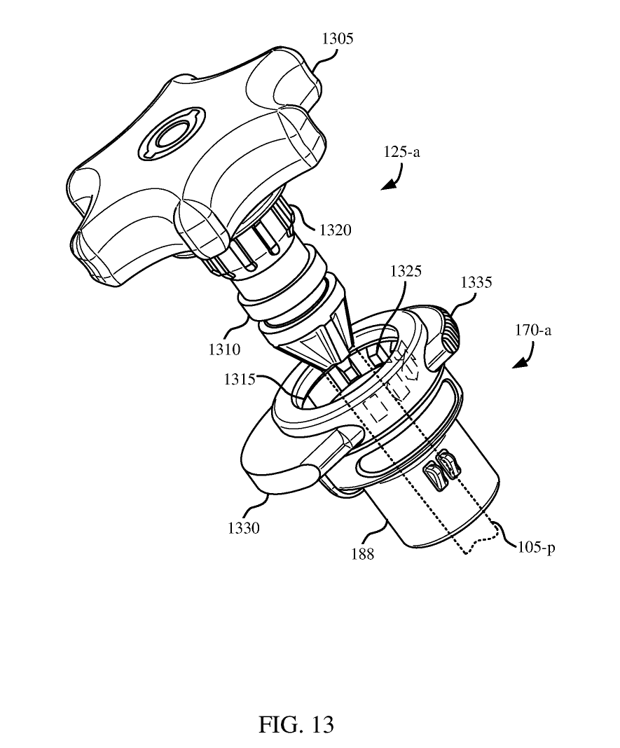

With reference to FIG. 13, an exploded assembly view of a cannula hub 125-a and the proximal end 188 of a handle assembly 170-a is illustrated in accordance with various embodiments. In the assembled and operable configuration, the cannula hub 125-a would be at least partially inserted into the handle assembly 170-a, but is shown here in an exploded view to illustrate various features of the cannula hub 125-a and handle assembly 170-a. As described with reference to FIG. 1, the cannula hub 125-a may be coupled with the proximal end 115 of a cannula 105-p (shown in phantom lines) and may be used to manipulate the cannula 105-p with respect to the handle assembly 170-a (e.g., rotation or axial translation). The cannula hub 125-a generally includes a proximal grip portion 1305 and a post portion 1310 extending from the grip portion 1305. The grip portion 1305 may include ridges, bumps, or any other similar features that facilitate gripping by a clinician for manipulation of the cannula 105-p. For example, a clinician may grasp the handle member 170-a within a palm and four fingers, and may rotate the cannula hub 125-a with the thumb.

The post portion 1310 is generally sized and configured to be inserted into a lumen 1315 of the handle assembly 170-a. In various embodiments, the post portion 1310 includes one or more features that interlock with the one or more features of the handle assembly 170-a to prevent or at least control movement of the cannula hub 125-a with respect to the handle assembly 170-a. For example, as the cannula hub 125-a is inserted into the lumen 1315 of the handle assembly 170-a, the post portion 1310 may click or snap into place, thereby preventing the cannula hub 125-a from falling back out of the handle assembly 170-a. To remove the cannula hub 125-a from the handle assembly 170-a, a release feature, such as release latch 1330 may be depressed or released.

In addition, the post portion 1310 may include one or more features to control the rotation of the cannula hub 125-a with respect to the handle assembly 170-a. For example, the post portion 1310 may include one or more detents 1320 that interlock with one or more detents 1325 that are disposed within the lumen 1315. The detents 1320 may be radially spaced around a partial or full circumference of the post portion 1310. Similarly, the detents 1325 within lumen 1315 may be radially spaced around a partial or full circumference of the lumen 1315. Although interlocking detents 1320, 1325 are described with reference to FIG. 13, it may be appreciated that any other feature or element that creates an interlocking engagement between the cannula hub 125-a and handle assembly 170-a may be used to control the rotation of the cannula hub 125-a with respect to the handle assembly 170-a. For example, ridges, posts, barbs, spring-loaded balls, or any other raised feature that interlocks with one or more corresponding raised or indented features to create a mechanical interlocking connection may be used.

In accordance with various embodiments, the detents 1320 may be selectively disengaged from the detents 1325 to permit relative rotation between the cannula hub 125-a and the handle assembly 170-a. For example, the detents 1325 may be disposed on a movable latch 1335 such that moving (e.g., by depressing) the latch 1335 inwards towards the center of lumen 1315 engages the detents 1325 with detents 1320, and moving the latch 1335 away from the center of the lumen 1315 disengages the detents 1325 from detents 1320. In some embodiments the latch 1335 may be biased (e.g., with a spring element) in either the engaged or disengaged configuration. For example, if the latch 1335 is biased in the disengaged configuration, then the detents 1325 will only engage with the detents 1320 if the latch 1335 is depressed (e.g., with the thumb of a clinician). Alternatively, if the latch 1335 is biased in the engaged configuration, then the detents 1325 will automatically engage with the detents 1320 as the post portion 1310 is inserted into the lumen 1315, and the cannula hub 125-a will be rotatable only after releasing the latch 1335 (e.g., by pushing the latch 1335 from the opposite side) to disengage the detents 1325, 1320 from each other. Engaging or disengaging the latch 1335 (or any other feature that moves the detents 1325, 1320) may include pressing, pulling, sliding, or screwing one or more elements.

It may be appreciated that other features, elements, and methods for selectively engaging the cannula hub 125-a with the handle assembly 170-a to control the rotation of the cannula hub 125-a may be used. For example, instead of moving the detents 1325 inward and outward to selectively engage with detents 1320, in some embodiments, the detents 1320 may be retracted and deployed from the post portion 1310 with a button, latch, or similar member coupled with the cannula hub 125-a. In alternative embodiments, instead of moving detents 1325, 1320 (or any other engagement members) with respect to each other for selective engagement, the post portion 1310 may include one or more features that create an interference fit with one or more corresponding features of the handle assembly 170-a. As such, rotating the cannula hub 125-a with respect to the handle assembly 170-a may include overcoming the resistance created by the interference fit. For example, the height of the detents 1325, 1320 may be selected such that, when aligned, there is an interference fit between them, but after a sufficient rotational force is imparted, the detents 1320, 1325 may snap or click past each other to allow rotation of the cannula hub 125-a. Alternatively, instead of using detents to create an interference fit, the post portion 1310 may include a rubber or other polymeric feature (e.g., an O-ring) that creates an interference fit with a corresponding feature within the lumen 1315, and this interference fit of the polymeric feature may be used exclusively to help control rotation of the cannula hub 125-a, or in combination with a selectively engaged locking hub feature.

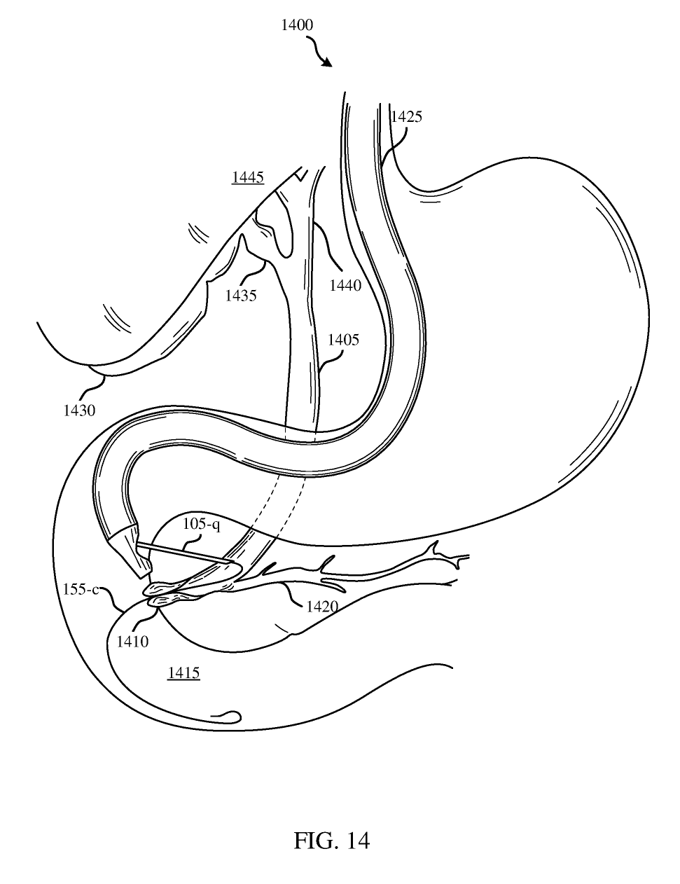

Embodiments of the present disclosure are now described in the context of a particular Endoscopic Ultrasound Guided Biliary Drainage (EUS-BD) procedure referred to as an Endoscopic Retrograde Cholangiopancreatography (ERCP) “Rendezvous” procedure. With reference to FIG. 14, a system 1400 for providing access to a body lumen within the pancreaticobiliary system is illustrated in accordance with various embodiments. The system 1400 may be examples of or include functionality of the systems or components described with reference to any of FIGS. 1-13. The illustrated portions of the pancreaticobiliary system include the common bile duct 1405, which drains bile from both the cystic duct 1435 (which drains from the gallbladder 1430) and the common hepatic duct 1440 (which drains from the liver 1445) into the duodenum 1415, where the bile mixes and reacts with digesting food. As shown, the common bile duct 1405 joins with the pancreatic duct 1420 at the ampulla of Vater 1410 (shown obstructed) before draining through the major duodenal papilla into the duodenum 1415.

Under a “Rendezvous” technique, a clinician may advance an endoscope 1425 (e.g., an EUS endoscope) into the lumen of a patient's duodenum 1415 to a position in which the bile ducts may be visualized (e.g., via endosonography). The clinician may then access the common bile duct 1405 by advancing a cannula 105-q from a working channel of the endoscope 1425, through the wall of the duodenum 1415 (i.e., trans-duodenally), and then through the wall of the common bile duct 1405. As described with reference to FIG. 3, the cannula 105-q may pierce the wall of the duodenum 1415 and the wall of the common bile duct 1405 by exposing the distal end of a sharpened stylet 135 (not shown for clarity) from the distal end 120 of the cannula 105-q.

Once at least the distal section 130 of the cannula 105-q is within the common bile duct 1405 (i.e., accessed the bile duct 1405), the clinician may then withdraw the stylet 135 from the cannula 105-q (or at least the distal section 130 of the cannula 105-q), thereby allowing the distal section 130 of the cannula 105-q to passively transition into a nonlinear shape within the common bile duct 1405, as described with reference to FIG. 2. In accordance with various embodiments, the nonlinear shape of the distal section 130 may then serve as an anchor to prevent the cannula 105-q from inadvertently falling back out of the bile duct 1405 through the access hole.

To verify that the cannula 105-q is actually within the common bile duct 1405, the clinician may use a syringe or vacuum to aspirate fluid from the body lumen and then verify that the aspirated fluid is bile (or any other confirmatory fluid depending on the target lumen or organ). If the common bile duct 1405 (or other target lumen) has not been properly accessed, the clinician may need to withdraw the cannula 105-q and re-straighten out the distal section 130 to pierce the proper lumen, as described with reference to FIGS. 12A-12B. Once proper placement within the common bile duct 1405 has been confirmed, the clinician may flush contrast fluid (i.e., fluid visible under fluoroscopy or any other imaging techniques) through the cannula 105-q and into the common bile duct 1405 to increase the visibility of the biliary lumens and verify proper cannulation of the bile duct 1405.

The clinician may then insert a guide wire 155-c into the cannula 105-q through the proximal end 115 and advance it distally towards the distal section 120. As described with reference to FIGS. 4A-4B and FIGS. 5A-5B, the clinician may then manipulate the distal end 120 of the cannula 105-q by rotating the cannula 105-q or by straightening out or curling up the distal section 130 of the cannula 105-q. In the case of the described “Rendezvous” procedure, the clinician may rotate (as described with reference to FIGS. 4A-4B) the distal section 130 of the cannula 105-q until the distal end 120 of the cannula 105-q is facing generally along the antegrade direction of flow of the common bile duct 1405 (i.e., in the direction of bile flow from the gallbladder 1430 to the duodenum 1415). Furthermore, the clinician may adjust the angle of sweep 215 (i.e., through straightening or curling) of the distal section 130 of the cannula 105-q by advancing a variable stiffness guide wire 155-c, as described with reference to FIGS. 5A-5B.

After the clinician has manipulated the distal section 130 of the cannula 105-q to the desired orientation through rotation or straightening, the guide wire 155-c may then be advanced distally from the distal end 120 of the cannula 105-q and through the bile duct 1405 and across the ampulla of Vater 1410 into the duodenum 1415. In some circumstances, the clinician may advance the cannula 105-q further into the bile duct 1405 over the guide wire 155-c (see FIGS. 6A-6B) so that the distal end 120 of the cannula 105-q is closer to the ampulla of Vater 1410 or luminal obstruction to be treated to provide additional support for crossing the luminal obstruction. When the guide wire 155-c has passed through the ampulla of Vater 1410 and well into the duodenum 1415, a “Rendezvous” procedure may be performed, wherein the EUS endoscope 1425 and the access system 1400 are withdrawn from the patient, leaving the guide wire 155-c in place. A side-viewing endoscope (e.g., duodenoscope) may then be passed into the duodenum 1415 adjacent the EUS-placed guide wire 155-c. The guide wire 155-c within the duodenum 1415 is grasped with a snare or forceps and withdrawn through the duodenoscope. Access to the common bile duct 1405 is then performed in reverse fashion over the guide wire 155-c, and a standard ERCP procedure can then be performed (e.g., open blocked ducts, break up or remove gallstones, insert stents, or endoscopic sphincterotomy). It should be noted that EUS-guided biliary access to the common bile duct 1405 is not limited to trans-duodenal access, as illustrated in FIG. 14. For example, access to the common bile duct 1405 may be achieved trans-gastrically, such that the cannula 105-q is advanced through the gastric wall and entry into the biliary system could involve the intrahepatic, extrahepatic, or common bile duct 1405.

In some embodiments, the system 1400 may be used to directly treat (e.g., antegrade stent delivery) the common bile duct 1405 directly through the access hole in the bile duct 1405 without requiring a “Rendezvous” procedure as described above. For example, as described with reference to FIGS. 7A-7D, the sheath 180 may be advanced into the common bile duct 1405 over the cannula 105-q and may serve as a stent delivery catheter, or serve as a conduit through which a stent delivery catheter may be passed through the access hole in the common bile duct 1405 to the blockage within the duct. In other examples, after advancing the sheath 180 within the common bile duct 1405, a separate stent delivery catheter may be advanced through the sheath 180 and into the common bile duct 1405. Alternatively, the guide wire 155-c may be advanced distally so that a substantial portion of the guide wire 155-c is placed in the duodenum 1415, and the cannula 105-q and sheath 180 may be withdrawn completely and exchanged, for example with a different sheath or a stent delivery catheter targeting stent placement across the bile duct 1405 including its papilla and ampulla, and its transition to the duodenum 1415.

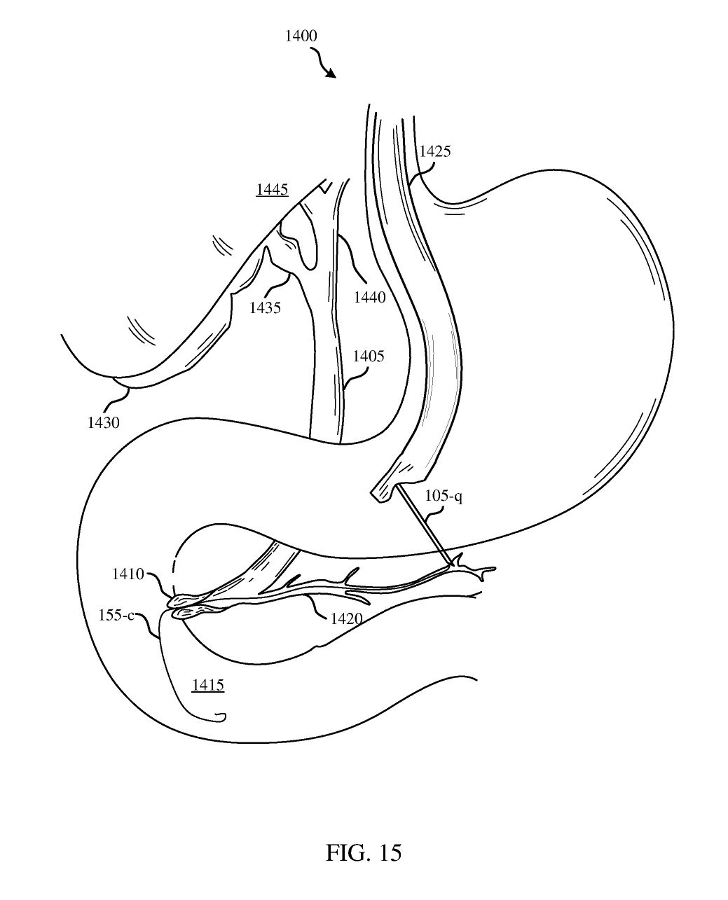

With reference to FIG. 15, the system 1400 may be used to directly access the pancreatic duct 1420 through the gastric wall. Such a procedure may be advantageous if the treatment site (e.g., obstruction) is located antegrade from where the common bile duct 1405 and pancreatic duct 1420 join. In addition, the system 1400 may be used to directly access the gallbladder 1430, the cystic duct 1435, the common hepatic duct 1440, or any other duct or organ within the pancreaticobiliary system. Moreover, the system 1400 may be used to access and treat (e.g., stent) any other lumen within the body such as those associated with the arterial system, the bronchial system, or the urinary system.

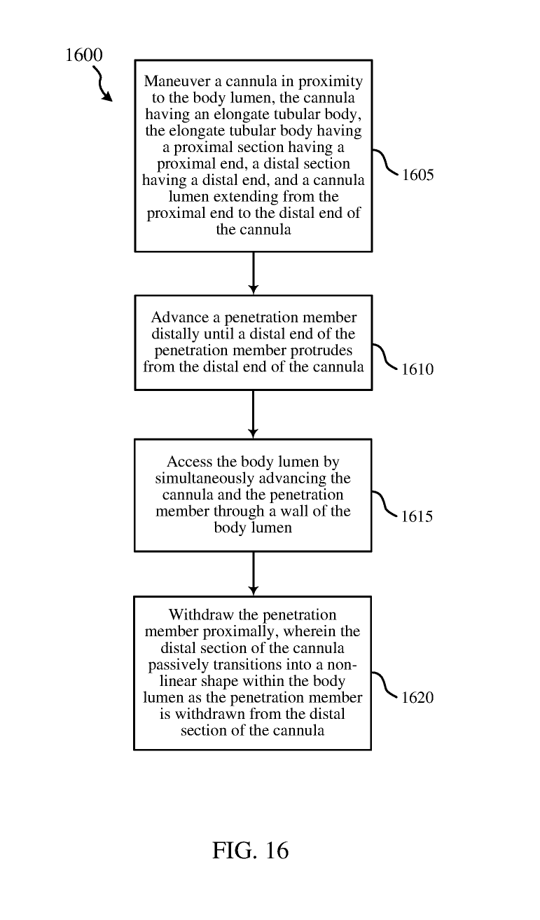

FIG. 16 shows a flowchart illustrating a method 1600 for accessing a body lumen in accordance with various aspects of the present disclosure. The steps of method 1600 may be performed with any of the systems or components described with reference to FIGS. 1-15 and may be an example of aspects of the particular procedure described with reference to FIG. 14. At block 1605, the method 1600 may include maneuvering a cannula 105 in proximity to a body lumen 305, the cannula 105 having an elongate tubular body, the elongate tubular body having a proximal section having a proximal end 115, a distal section 130 having a distal end 120, and a cannula lumen 110 extending from the proximal end 115 to the distal end 120 of the cannula 105. As described with reference to FIG. 1, maneuvering the cannula 105 may include advancing the cannula 105 through the working channel of an endoscope or through an external sheath 180.

At block 1610, the method 1600 may also include, but is not limited to, advancing a penetration member (e.g., a stylet 135) distally until a distal end 145 of the penetration member protrudes from the distal end 120 of the cannula 105. In accordance with various embodiments, this step may be described as exposing the stylet 135. As described with reference to FIGS. 11A-11C, the distal end 145 of the stylet 135 may be sharpened and include one or more beveled features to form a variety of sharpened configurations.

At block 1615, the method 1600 may also include accessing the body lumen 305 by simultaneously advancing the cannula 105 and the penetration member (e.g., stylet 135) through a wall 310 of the body lumen 305, as described with reference to FIG. 3. As described with reference to FIGS. 10A-10B, the cannula 105 or stylet 135 may include one or more features to reduce the force required to pierce the stylet 135 and cannula 105 through the wall 310 of a body lumen 305.

At block 1620, the method 1600 may also include withdrawing the penetration member proximally, such that the distal section 130 of the cannula 105 passively transitions into a nonlinear shape within the body lumen 305 as the penetration member is withdrawn from the distal section 130 of the cannula 105, as described with reference to FIGS. 4A-4B.

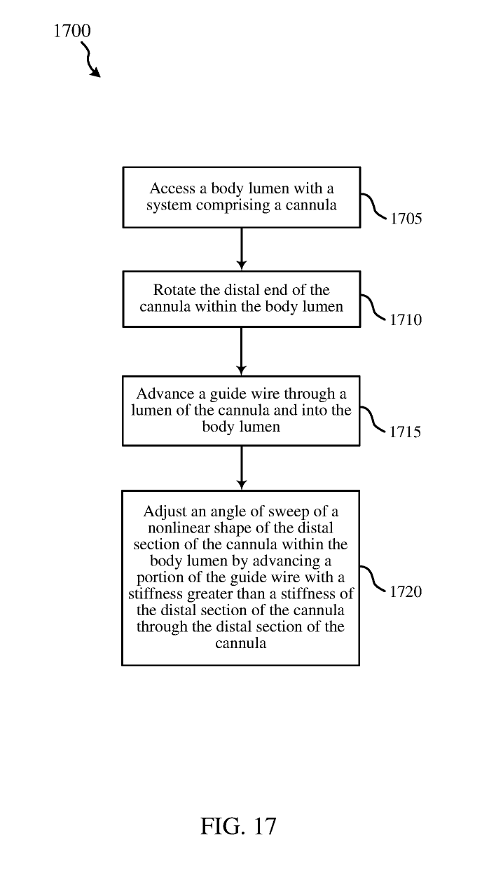

FIG. 17 shows a flowchart illustrating a method 1700 for accessing a body lumen in accordance with various aspects of the present disclosure. The steps of method 1700 may be performed with any of the systems or components described with reference to FIGS. 1-15 and may be an example of aspects of the particular procedure described with reference to FIG. 14.

At block 1705, the method 1700 may include accessing a body lumen 305 with a system comprising a cannula 105, which may be an example of aspects of method 1600 described with reference to FIG. 16.

At block 1710, the method 1700 may include rotating a distal end 120 of the cannula 105 within the body lumen 305, as described with reference to FIGS. 4A-4B. In accordance with various embodiments, rotating a distal end 120 of the cannula 105 may include rotating a cannula hub 125 with respect to a handle assembly 170. Moreover, in certain embodiments, rotating the distal end 120 may also include selectively engaging the cannula hub 125 with the handle assembly 170 for controlled rotation of the cannula 105, as described with reference to FIG. 13.

At block 1715, the method 1700 may including advancing a guide wire 155 through the cannula lumen 110 and into the body lumen 305.

At block 1720, the method 1700 may include adjusting an angle of sweep 215 of the nonlinear shape of the distal section 130 of the cannula 105 by advancing a portion 165 of the guide wire 155 with a stiffness greater than a stiffness of the distal section 130 of the cannula 105 through the distal section 130 of the cannula 105, as described with reference to FIGS. 5A-5B.

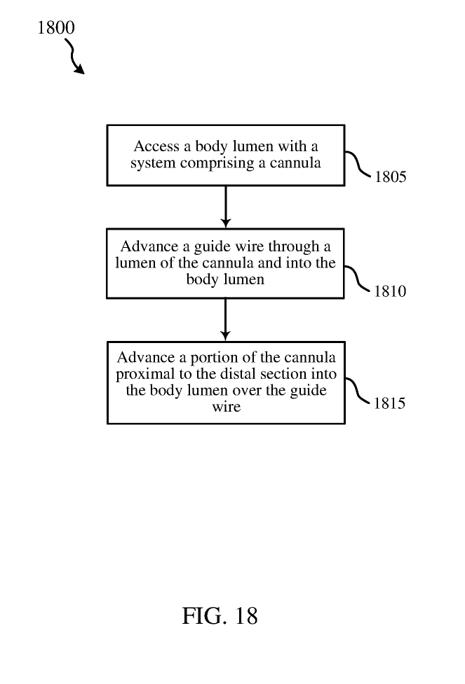

FIG. 18 shows a flowchart illustrating a method 1800 for accessing a body lumen in accordance with various aspects of the present disclosure. The steps of method 1800 may be performed with any of the systems or components described with reference to FIGS. 1-15 and may be an example of aspects of the particular procedure described with reference to FIG. 14.

At block 1805, the method 1800 may include accessing a body lumen 305 with a system comprising a cannula 105, which may be an example of aspects of method 1600 described with reference to FIG. 16.

At block 1810, the method 1800 may including advancing a guide wire 155 through the cannula lumen 110 and into the body lumen 305.

At block 1815, the method 1800 may include advancing a portion 605 of the cannula 105 proximal to the distal section 130 into the body lumen 305 over the guide wire 155, as described with reference to FIGS. 6A-6B.

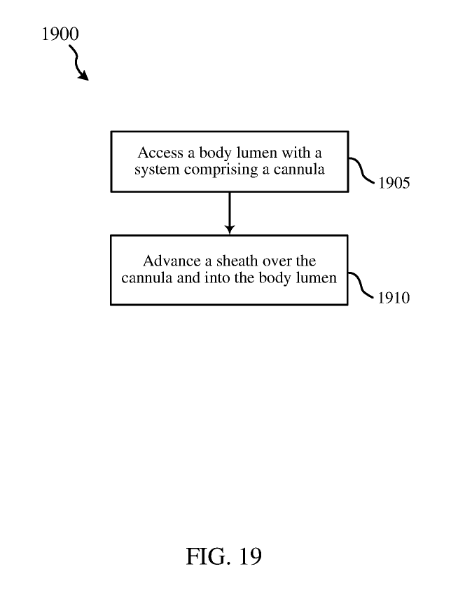

FIG. 19 shows a flowchart illustrating a method 1900 for accessing a body lumen in accordance with various aspects of the present disclosure. The steps of method 1900 may be performed with any of the systems or components described with reference to FIGS. 1-15 and may be an example of aspects of the particular procedure described with reference to FIG. 14.

At block 1905, the method 1900 may include accessing a body lumen 305 with a system comprising a cannula 105, which may be an example of aspects of method 1600 described with reference to FIG. 16.

At block 1910, the method 1900 may including advancing a sheath 180 over the cannula 105 and into the body lumen 305, as described with reference to FIGS. 7A-7D. In some examples, the sheath 180 serves as a stent delivery catheter for antegrade treatment of the body lumen 305 through the pierced access hole. Alternatively, a separate stent delivery catheter may be advanced through the sheath 180 once it is placed within the body lumen 305. In yet other examples, the sheath 180 and cannula 105 are completely withdrawn, leaving the guide wire 155 within the body lumen 305, and a separate stent delivery catheter is advanced over the guide wire 155 and into the body lumen 305.

It should be noted that these methods describe possible implementation, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

While several embodiments of the present disclosure have been described and illustrated herein, those of ordinary skill in the art will readily envision a variety of other means or structures for performing the functions or obtaining the results or one or more of the advantages described herein, and each of such variations or modifications is deemed to be within the scope of the present disclosure. More generally, those skilled in the art will readily appreciate that all parameters, dimensions, materials, and configurations described herein are meant to be exemplary and that the actual parameters, dimensions, materials, or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, or method described herein. In addition, any combination of two or more such features, systems, articles, materials, kits, or methods, if such features, systems, articles, materials, kits, or methods are not mutually inconsistent, is included within the scope of the present disclosure.

All definitions, as defined and used herein, should be understood to control over dictionary definitions, definitions in documents incorporated by reference, or ordinary meanings of the defined terms.

The indefinite articles “a” and “an,” as used herein in the specification and in the claims, unless clearly indicated to the contrary, should be understood to mean “at least one.” Also, as used herein, including in the claims, “or” as used in a list of items (for example, a list of items prefaced by a phrase such as “at least one of” or “one or more”) indicates an inclusive list such that, for example, a list of at least one of A, B, or C means A or B or C or AB or AC or BC or ABC (i.e., A and B and C).

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed is:

1. A system for providing access to a body lumen, comprising:
a cannula having an elongate tubular body, the elongate tubular body having a proximal section having a proximal end, a distal section having a distal end, and a cannula lumen extending from the proximal end to the distal end of the cannula, wherein the distal section of the cannula is configured to penetrate a wall of the body lumen and passively transition to a nonlinear shape within the body lumen as an elongate member is withdrawn from the distal section of the cannula lumen.

2. The system of claim 1, wherein an orientation of the distal end of the cannula is adjustable within the body lumen.

3. The system of claim 1, wherein an angle of sweep of the nonlinear shape is adjustable within the body lumen.

4. The system of claim 1, wherein the cannula is configured to transmit torque from the proximal end of the cannula to the distal end of the cannula to rotate the distal end of the cannula within the body lumen.

5. The system of claim 1, wherein the nonlinear shape is configured to retain the distal section of the cannula within the body lumen.

6. The system of claim 1, wherein the elongate member comprises a sharpened distal end and is configured to protrude from the distal end of the cannula to penetrate the wall of the body lumen.

7. The system of claim 1, wherein the distal end of the cannula is blunt.

8. The system of claim 1, wherein the distal end of the cannula is sharpened.

9. The system of claim 1, further comprising a guide wire sized to advance through the cannula lumen and having at least a first portion with a first stiffness and a second portion with a second stiffness, wherein the second stiffness is greater than the first stiffness.

10. The system of claim 9, wherein the first stiffness is less than a stiffness of the distal section of the cannula, and wherein the second stiffness is greater than the stiffness of the distal section of the cannula such that as the second portion of the guide wire is advanced and withdrawn past the distal section of the cannula, an angle of sweep of the nonlinear shape is adjusted.

11. The system of claim 1, further comprising a guide wire sized to advance through the cannula lumen and having a portion configured to transition the distal section of the cannula from the nonlinear shape into a substantially linear shape to facilitate removal of the distal section of the cannula from the body lumen.

12. The system of claim 1, wherein the cannula includes a plurality of apertures disposed along a length of the distal section.

13. The system of claim 12, wherein the plurality of apertures comprises a first row of apertures extending longitudinally along the length of the distal section and a second row of apertures extending longitudinally along the length of the distal section and disposed diametrically opposite the first row of apertures.

14. The system of claim 12, wherein the plurality of apertures comprises dogbone apertures having a central portion and an end portion on each end of the central portion, the central portion oriented substantially perpendicular to a longitudinal axis defined by the cannula lumen and each end portion oriented substantially perpendicular to the central portion.

15. The system of claim 12, wherein the plurality of apertures comprises rectangular apertures oriented substantially perpendicular to a longitudinal axis defined by the cannula lumen.

16. The system of claim 12, wherein the plurality of apertures comprises apertures having a plurality of curved features.

17. The system of claim 1, wherein the nonlinear shape is pre-defined by a heat treatment process.

18. The system of claim 1, wherein an angle of sweep of the nonlinear shape is between 0 and 480 degrees.

19. The system of claim 1, wherein a centerline radius of curvature of the nonlinear shape is between 0.20 inches and 0.65 inches.

20. The system of claim 1, wherein an outer diameter of the cannula is between 0.02025 inches and 0.065 inches.

21. The system of claim 1, wherein the body lumen is a biliary lumen.

22. A system for providing access to a body lumen, comprising:
a handle having a proximal end, a distal end, and a handle lumen extending from the proximal end to the distal end of the handle;
a cannula removably disposed within the handle lumen, the cannula having an elongate tubular body having a proximal section having a proximal end, a distal section having a distal end, and a cannula lumen extending from the proximal end to the distal end of the cannula;
a penetration member sized to advance through the cannula lumen, wherein the distal section of the cannula is configured to passively transition to a nonlinear shape within the body lumen as the penetration member is withdrawn from the cannula lumen; and
a cannula hub coupled with the proximal end of the cannula, the cannula hub configured to selectively engage with the proximal end of the handle to selectively lock rotational movement of the cannula with respect to the handle.

23. The system of claim 22, further comprising a sheath coupled with the distal end of the handle and having a sheath lumen sized to slidably accept the cannula.

24. A method of accessing a body lumen, comprising:
maneuvering a cannula in proximity to the body lumen, the cannula having an elongate tubular body, the elongate tubular body having a proximal section having a proximal end, a distal section having a distal end, and a cannula lumen extending from the proximal end to the distal end of the cannula;
advancing a penetration member distally until a distal end of the penetration member protrudes from the distal end of the cannula;
accessing the body lumen by simultaneously advancing the cannula and the penetration member through a wall of the body lumen; and
withdrawing the penetration member proximally, wherein the distal section of the cannula passively transitions into a nonlinear shape within the body lumen as the penetration member is withdrawn from the distal section of the cannula.

25. The method of claim 24, further comprising:
rotating the distal end of the cannula within the body lumen.

26. The method of claim 24, further comprising:
advancing a guide wire through the cannula lumen and into the body lumen.

27. The method of claim 26, further comprising:
adjusting an angle of sweep of the nonlinear shape by advancing a portion of the guide wire with a stiffness greater than a stiffness of the distal section of the cannula through the distal section of the cannula.

28. The method of claim 24, wherein the nonlinear shape prevents the cannula from falling out of the body lumen.

29. The method of claim 24, further comprising:
advancing a proximal section of the cannula located proximal to the distal section into the body lumen.

30. The method of claim 24, further comprising:
advancing a sheath into the body lumen.

31. The method of claim 30, wherein the sheath is advanced over the cannula into the body lumen.

32. The method of claim 30, wherein the sheath is advanced over a guide wire into the body lumen after the cannula has been withdrawn from the body lumen.

33. The method of claim 30, wherein the sheath comprises a stent delivery catheter.
